(19) United States
(12) Patent Application Publication
(10) Pub. No.: US 2025/0255459 A1
(43) Pub. Date: Aug. 14, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Yasuhiko MORIMOTO, Kanagawa (JP)

(73) Assignee: FUJIFILM Corporation, Tokyo (JP)

(21) Appl. No.: 19/193,933

(22) Filed: Apr. 29, 2025

Related U.S. Application Data
(63) Continuation of application No. PCT/JP2023/036269, filed on Oct. 4, 2023.

(30) Foreign Application Priority Data
Nov. 4, 2022 (JP) 2022-177613

(51) Int. Cl. A61B 1/00; A61B 1/005; A61B 1/05; A61B 1/273; G06T 7/00

(52) U.S. Cl. CPC A61B 1/000094; A61B 1/000096; A61B 1/00045; A61B 1/005; A61B 1/05; A61B 1/273; G06T 7/0012; G06T 2207/10068; G06T 2207/20084; G06T 2207/30028

(57) ABSTRACT

A medical support device includes a processor, in which the processor is configured to acquire intestinal tract direction-related information related to an intestinal tract direction of a duodenum into which an endoscope scope is inserted, based on geometrical characteristic information capable of specifying a geometrical characteristic of the duodenum; and output the intestinal tract direction-related information.

### CROSS-REFERENCE TO RELATED APPLICATIONS

This application is a continuation application of International Application No. PCT/JP2023/036269, filed Oct. 4, 2023, the disclosure of which is incorporated herein by reference in its entirety. Further, this application claims priority from Japanese Patent Application No. 2022-177613, filed Nov. 4, 2022, the disclosure of which is incorporated herein by reference in its entirety.

### BACKGROUND

### 1. Technical Field

The technology of the present disclosure relates to a medical support device, an endoscope, a medical support method, and a program.

### 2. Description of the Related Art

JP2020-62218A discloses a learning apparatus comprising an acquisition unit that acquires a plurality of pieces of information in which an image of a duodenal Vater's papilla of a bile duct and information indicating a cannulation method, which is a method of inserting a catheter into the bile duct, are associated with each other; a learning unit that performs machine learning using the information indicating the cannulation method as training data based on the image of the duodenal Vater's papilla of the bile duct; and a storage unit that stores a result of the machine learning performed by the learning unit and the information indicating the cannulation method in association with each other.

### SUMMARY

One embodiment according to the technology of the present disclosure provides a medical support device, an endoscope, a medical support method, and a program that allow a user to easily grasp to what extent a posture of an endoscope scope deviates with respect to an intestinal tract direction of a duodenum.

A first aspect according to the technology of the present disclosure is a medical support device comprising a processor, in which the processor is configured to acquire intestinal tract direction-related information related to an intestinal tract direction of a duodenum into which an endoscope scope is inserted, based on geometrical characteristic information capable of specifying a geometrical characteristic of the duodenum; and output the intestinal tract direction-related information.

A second aspect according to the technology of the present disclosure is the medical support device according to the first aspect, in which the intestinal tract direction-related information includes deviation amount information indicating a deviation amount between a posture of the endoscope scope and the intestinal tract direction.

A third aspect according to the technology of the present disclosure is the medical support device according to the first aspect or the second aspect, in which the geometrical characteristic information includes an intestinal wall image obtained by imaging an intestinal wall of the duodenum with a camera provided in the endoscope scope, and the processor is configured to acquire the intestinal tract direction-related information by executing first image recognition processing on the intestinal wall image.

A fourth aspect according to the technology of the present disclosure is the medical support device according to any one of the first to third aspects, in which the outputting of the intestinal tract direction-related information includes displaying the intestinal tract direction-related information on a first screen.

A fifth aspect according to the technology of the present disclosure is the medical support device according to any one of the first to fourth aspects, in which the intestinal tract direction-related information includes first direction information capable of specifying a first direction intersecting the intestinal tract direction at a predetermined angle.

A sixth aspect according to the technology of the present disclosure is the medical support device according to the fifth aspect, in which the first direction information is obtained by executing second image recognition processing on an intestinal wall image obtained by imaging an intestinal wall of the duodenum with a camera provided in the endoscope scope.

A seventh aspect according to the technology of the present disclosure is the medical support device according to the sixth aspect, in which the first direction information is information obtained with a degree of certainty equal to or greater than a threshold value by performing image recognition processing using an AI method as the second image recognition processing.

An eighth aspect according to the technology of the present disclosure is the medical support device according to any one of the first to seventh aspects, in which the processor is configured to acquire posture information capable of specifying a posture of the endoscope scope in a state in which the endoscope scope is inserted into the duodenum, the intestinal tract direction-related information includes posture adjustment support information for supporting adjustment of the posture, and the posture adjustment support information is information determined based on a deviation amount between the intestinal tract direction and the posture specified from the posture information.

A ninth aspect according to the technology of the present disclosure is the medical support device according to any one of the first to eighth aspects, in which the intestinal tract direction-related information includes condition information indicating a condition for changing a posture of the endoscope scope to match an optical axis direction of a camera provided in the endoscope scope with a second direction intersecting the intestinal tract direction at a predetermined angle.

A tenth aspect according to the technology of the present disclosure is the medical support device according to the ninth aspect, in which the condition includes an operation condition related to an operation performed on the endoscope scope to match the optical axis direction with the second direction.

An eleventh aspect according to the technology of the present disclosure is the medical support device according to any one of the first to tenth aspects, in which, in a case where an optical axis direction of a camera provided in the endoscope scope matches a third direction intersecting the intestinal tract direction at a predetermined angle, the intestinal tract direction-related information includes notification information for notifying that the optical axis direction matches the third direction.

A twelfth aspect according to the technology of the present disclosure is the medical support device according to any one of the first to eleventh aspects, in which the processor is configured to detect a duodenal papilla region by executing third image recognition processing on an intestinal wall image obtained by imaging an intestinal wall of the duodenum with a camera provided in the endoscope scope; display the duodenal papilla region on a second screen; and display, on the second screen, papilla orientation information indicating an orientation of the duodenal papilla region, the papilla orientation information being obtained based on the intestinal tract direction-related information.

A thirteenth aspect according to the technology of the present disclosure is the medical support device according to any one of the first to twelfth aspects, in which the processor is configured to detect a duodenal papilla region by executing fourth image recognition processing on an intestinal wall image obtained by imaging an intestinal wall of the duodenum with a camera provided in the endoscope scope; display the duodenal papilla region on a third screen; and display, on the third screen, running direction information indicating a running direction of a duct leading to an opening of the duodenal papilla region, the running direction information being obtained based on the intestinal tract direction-related information.

A fourteenth aspect according to the technology of the present disclosure is the medical support device according to the thirteenth aspect, in which the duct is a bile duct or a pancreatic duct.

A fifteenth aspect according to the technology of the present disclosure is the medical support device according to any one of the first to fourteenth aspects, in which the geometrical characteristic information includes depth information indicating a depth of the duodenum, and the intestinal tract direction-related information is acquired based on the depth information.

A sixteenth aspect according to the technology of the present disclosure is an endoscope comprising the medical support device according to any one of the first to fifteenth aspects, and the endoscope scope.

A seventeenth aspect according to the technology of the present disclosure is a medical support method comprising acquiring intestinal tract direction-related information related to an intestinal tract direction of a duodenum into which an endoscope scope is inserted, based on geometrical characteristic information capable of specifying a geometrical characteristic of the duodenum; and outputting the intestinal tract direction-related information.

An eighteenth aspect according to the technology of the present disclosure is a program causing a computer to execute processing comprising acquiring intestinal tract direction-related information related to an intestinal tract direction of a duodenum into which an endoscope scope is inserted, based on geometrical characteristic information capable of specifying a geometrical characteristic of the duodenum; and outputting the intestinal tract direction-related information.

### BRIEF DESCRIPTION OF THE DRAWINGS

FIG. 1 is a conceptual diagram showing an example of an aspect in which a duodenoscope system is used.

FIG. 2 is a conceptual diagram showing an example of an overall configuration of the duodenoscope system.

FIG. 3 is a block diagram showing an example of a hardware configuration of an electrical system of the duodenoscope system.

FIG. 4 is a conceptual diagram showing an example of an aspect in which a duodenoscope is used.

FIG. 5 is a block diagram showing an example of a hardware configuration of an electrical system of an image processing device.

FIG. 6 is a conceptual diagram showing an example of the correlation between an endoscope scope, a duodenoscope body, an image acquisition unit, an image recognition unit, and a derivation unit.

FIG. 7 is a conceptual diagram showing an example of the correlation between the display device, the image acquisition unit, the image recognition unit, the derivation unit, and a display control unit.

FIG. 8 is a flowchart showing an example of a flow of medical support processing.

FIG. 9 is a conceptual diagram showing an example of the correlation between the endoscope scope, the duodenoscope body, the image acquisition unit, the image recognition unit, and the derivation unit.

FIG. 10 is a conceptual diagram showing an example of the correlation between the endoscope scope, the duodenoscope body, the image acquisition unit, the image recognition unit, and the derivation unit.

FIG. 11 is a conceptual diagram showing an example of the correlation between the display device, the image recognition unit, the derivation unit, and the display control unit.

FIG. 12 is a conceptual diagram showing an example of the correlation between the endoscope scope, the image acquisition unit, the image recognition unit, and the derivation unit.

FIG. 13 is a conceptual diagram showing an example of the correlation between the display device, the image recognition unit, the derivation unit, and the display control unit.

FIG. 14 is a conceptual diagram showing an example of the correlation between the display device, the derivation unit, and the display control unit.

FIG. 15 is a conceptual diagram showing an example of the correlation between the endoscope scope, the duodenoscope body, the image acquisition unit, the image recognition unit, and the derivation unit.

FIG. 16 is a conceptual diagram showing an example of the correlation between the display device, the image recognition unit, the derivation unit, and the display control unit.

FIG. 17 is a conceptual diagram showing an example of an aspect in which the endoscope scope is made to directly face a papilla.

FIG. 18 is a conceptual diagram showing an example of the correlation between the endoscope scope, the duodenoscope body, the image acquisition unit, the image recognition unit, and the derivation unit.

FIG. 19 is a conceptual diagram showing an example of the correlation between the endoscope scope, the duodenoscope body, the image acquisition unit, the image recognition unit, and the derivation unit.

FIG. 20 is a conceptual diagram showing an example of the correlation between the display device, the image recognition unit, the derivation unit, and the display control unit.

FIG. 21 is a conceptual diagram showing an example of the correlation between the endoscope scope, the duodenoscope body, the image acquisition unit, the image recognition unit, and the derivation unit.

FIG. 22 is a conceptual diagram showing an example of the correlation between the display device, the image recognition unit, the derivation unit, and the display control unit.

FIG. 23 is a conceptual diagram showing an example of the correlation between the display device, the image recognition unit, the derivation unit, and the display control unit.

FIG. 24 is a conceptual diagram showing an example of the correlation between the endoscope scope, the image acquisition unit, the image recognition unit, and the derivation unit.

FIG. 25 is a conceptual diagram showing an example of the correlation between the display device, the derivation unit, and the display control unit.

FIG. 26 is a flowchart showing an example of a flow of medical support processing.

FIG. 27 is a conceptual diagram showing an example of the correlation between the display device, the derivation unit, and the display control unit.

FIG. 28 is a conceptual diagram showing an example of the correlation between the endoscope scope, the image acquisition unit, the image recognition unit, and the derivation unit.

FIG. 29 is a conceptual diagram showing an example of the correlation between the display device, the derivation unit, and the display control unit.

FIG. 30 is a conceptual diagram showing an example of the correlation between the endoscope scope, the image acquisition unit, the image recognition unit, and the derivation unit.

FIG. 31 is a conceptual diagram showing an example of the correlation between the endoscope scope, the image acquisition unit, the image recognition unit, and the derivation unit.

FIG. 32 is a conceptual diagram showing an example of the correlation between the endoscope scope, the image acquisition unit, the image recognition unit, and the derivation unit.

FIG. 33 is a conceptual diagram showing an example of the correlation between the endoscope scope, the image acquisition unit, the image recognition unit, and the derivation unit.

FIG. 34 is a conceptual diagram showing an example of the correlation between the display device, the derivation unit, and the display control unit.

FIG. 35 is a conceptual diagram showing an example of the correlation between the endoscope scope, the image acquisition unit, the image recognition unit, and the derivation unit.

FIG. 36 is a conceptual diagram showing an example of the correlation between the display device, the derivation unit, and the display control unit.

FIG. 37 is a conceptual diagram showing an example of the correlation between the display device, the derivation unit, and the display control unit.

### DETAILED DESCRIPTION

Hereinafter, examples of embodiments of a medical support device, an endoscope, a medical support method, and a program according to the technology of the present disclosure will be described with reference to the accompanying drawings.

First, terms used in the following description will be described.

CPU is an abbreviation for “central processing unit”. GPU is an abbreviation for “graphics processing unit”. RAM is an abbreviation for random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. FIFO is an abbreviation for “first in, first out”. ERCP is an abbreviation for “endoscopic retrograde cholangio-pancreatography”. ToF is an abbreviation for “time of flight”.

### First Embodiment

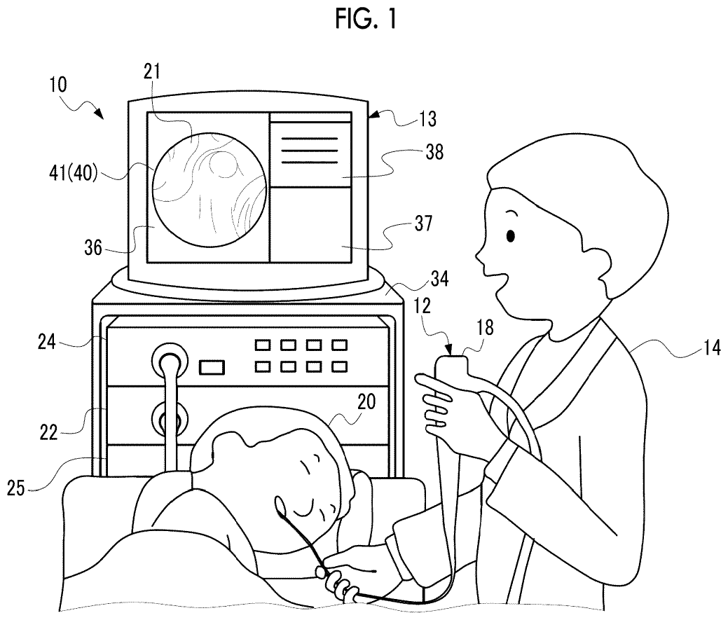

For example, as shown in FIG. 1, a duodenoscope system 10 comprises a duodenoscope 12 and a display device 13. The duodenoscope 12 is used by a doctor 14 in endoscopy. The duodenoscope 12 is communicably connected to a communication device (not shown), and information obtained by the duodenoscope 12 is transmitted to the communication device. The communication device receives the information transmitted from the duodenoscope 12 and performs processing using the received information (for example, the processing of recording the information on an electronic medical record or the like).

The duodenoscope 12 comprises an endoscope scope 18. The duodenoscope 12 is a device for performing medical care on an observation target 21 (for example, a duodenum) included in a body of a subject 20 (for example, a patient) using the endoscope scope 18. The observation target 21 is a target observed by the doctor 14. The endoscope scope 18 is inserted into the body of the subject 20. The duodenoscope 12 causes the endoscope scope 18 inserted into the body of the subject 20 to image the observation target 21 inside the body of the subject 20, and performs various medical treatments on the observation target 21 as necessary. The duodenoscope 12 is an example of an “endoscope” according to the technology of the present disclosure.

The duodenoscope 12 images the inside of the body of the subject 20 to acquire an image showing an aspect of the inside of the body and outputs the image. In the present embodiment, the duodenoscope 12 is an endoscope having an optical imaging function of irradiating the inside of the body with light to image the light reflected by the observation target 21.

The duodenoscope 12 comprises a control device 22, a light source device 24, and an image processing device 25. The control device 22 and the light source device 24 are installed in a wagon 34. A plurality of tables are provided in the wagon 34 in a vertical direction, and the image processing device 25, the control device 22, and the light source device 24 are installed from a lower table to an upper table. In addition, the display device 13 is installed on the uppermost table in the wagon 34.

The control device 22 is a device that controls the entire duodenoscope 12. In addition, the image processing device 25 is a device that performs image processing on the image captured by the duodenoscope 12 under the control of the control device 22.

The display device 13 displays various types of information including an image (for example, an image subjected to image processing by the image processing device 25). An example of the display device 13 includes a liquid-crystal display or an EL display. In addition, a tablet terminal with a display may be used instead of the display device 13 or together with the display device 13.

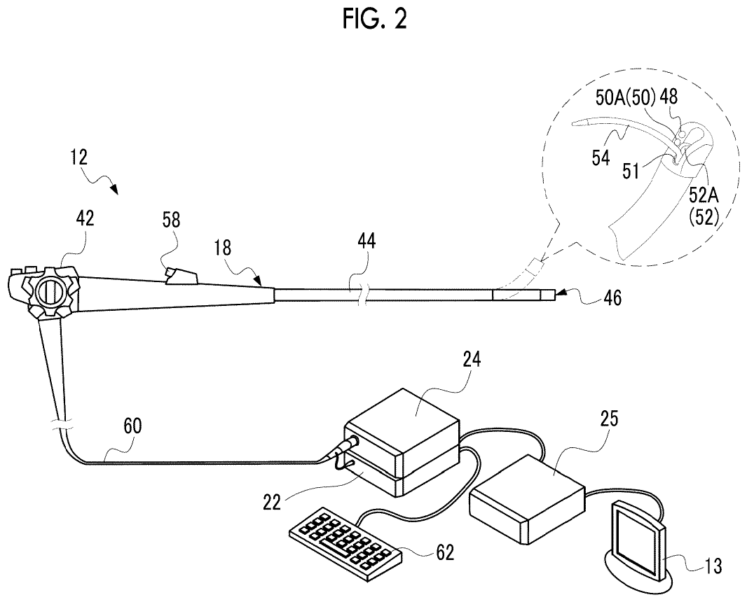

A plurality of screens are displayed side by side on the display device 13. In the example shown in FIG. 1, screens 36, 37, and 38 are shown. An endoscopic image 40 obtained by the duodenoscope 12 is displayed on the screen 36. The observation target 21 is captured in the endoscopic image 40. The endoscopic image 40 is an image obtained by imaging the observation target 21 with a camera 48 (see FIG. 2) provided in the endoscope scope 18 inside the body of the subject 20. An example of the observation target 21 includes an intestinal wall of a duodenum. In the following, for convenience of description, an intestinal wall image 41, which is an endoscopic image 40 in which the intestinal wall of the duodenum is imaged, is described as an example of the observation target 21. In addition, the duodenum is merely an example, and any region that can be imaged by the duodenoscope 12 may be used. For example, an esophagus or a stomach is given as an example of the region that can be imaged by the duodenoscope 12. The intestinal wall image 41 is an example of the “intestinal wall image” and the “geometrical characteristic information” according to the technology of the present disclosure.

A moving image including a plurality of frames of the intestinal wall images 41 is displayed on the screen 36. That is, the plurality of frames of intestinal wall images 41 are displayed on the screen 36 at a predetermined frame rate (for example, several tens of frames/sec).

As shown in FIG. 2 as an example, the duodenoscope 12 comprises an operating part 42 and an insertion part 44. The insertion part 44 is partially bent by operating the operating part 42. The insertion part 44 is inserted while being bent according to the shape of the observation target 21 (for example, the shape of the stomach) in response to the operation of the operating part 42 by the doctor 14.

The camera 48, an illumination device 50, a treatment opening 51, and an elevating mechanism 52 are provided at a distal end part 46 of the insertion part 44. The camera 48 and the illumination device 50 are provided on a side surface of the distal end part 46. That is, the duodenoscope 12 serves as a side-viewing scope. Accordingly, the intestinal wall of the duodenum is easily observed.

The camera 48 is a device that acquires the intestinal wall image 41 as a medical image by imaging the inside of the body of the subject 20. An example of the camera 48 includes a CMOS camera. However, this is merely an example, and the camera 48 may be other types of cameras such as CCD cameras. The camera 48 is an example of the “camera” according to the technology of the present disclosure.

The illumination device 50 has an illumination window 50A. The illumination device 50 emits light through the illumination window 50A. Examples of the type of the light emitted from the illumination device 50 include visible light (for example, white light) and invisible light (for example, near-infrared light). In addition, the illumination device 50 emits special light through the illumination window 50A. Examples of the special light include light for BLI and/or light for LCI. The camera 48 images the inside of the body of the subject 20 using an optical method in a state in which the inside of the body of the subject 20 is irradiated with light by the illumination device 50.

The treatment opening 51 is used as a treatment tool protruding port through which a treatment tool 54 is made to protrude from the distal end part 46, a suction port for suctioning, for example, blood and body waste, and a delivery port for sending out a fluid.

The treatment tool 54 protrudes from the treatment opening 51 in response to the operation of the doctor 14. The treatment tool 54 is inserted into the insertion part 44 from a treatment tool insertion port 58. The treatment tool 54 passes through the inside of the insertion part 44 through the treatment tool insertion port 58 and protrudes from the treatment opening 51 into the body of the subject 20. In the example shown in FIG. 2, a cannula protrudes from the treatment opening 51 as the treatment tool 54. The cannula is merely an example of the treatment tool 54, and other examples of the treatment tool 54 include a papillotomy knife and a snare.

The elevating mechanism 52 changes a protruding direction of the treatment tool 54 protruding from the treatment opening 51. The elevating mechanism 52 comprises a guide 52A, and the guide 52A rises with respect to the protruding direction of the treatment tool 54, so that the protruding direction of the treatment tool 54 is changed along the guide 52A. Accordingly, it is easy to protrude the treatment tool 54 toward the intestinal wall. In the example shown in FIG. 2, the protruding direction of the treatment tool 54 is changed to a direction perpendicular to a traveling direction of the distal end part 46 by the elevating mechanism 52. The elevating mechanism 52 is operated by the doctor 14 using the operating part 42. Accordingly, the degree of change in the protruding direction of the treatment tool 54 is adjusted.

The endoscope scope 18 is connected to the control device 22 and the light source device 24 through a universal cord 60. The display device 13 and a receiving device 62 are connected to the control device 22. The receiving device 62 receives an instruction from a user (for example, the doctor 14) and outputs the received instruction as an electric signal. In the example shown in FIG. 2, a keyboard is given as an example of the receiving device 62. However, this is merely an example, and the receiving device 62 may be, for example, a mouse, a touch panel, a foot switch, and/or a microphone.

The control device 22 controls the entire duodenoscope 12. For example, the control device 22 controls the light source device 24 or transmits and receives various signals to and from the camera 48. The light source device 24 emits light under the control of the control device 22 and supplies the light to the illumination device 50. A light guide is provided in the illumination device 50, and the light supplied from the light source device 24 is emitted from the illumination windows 50A and 50B through the light guide. The control device 22 causes the camera 48 to execute the imaging, acquires the intestinal wall image 41 (see FIG. 1) from the camera 48, and outputs the intestinal wall image 41 to a predetermined output destination (for example, the image processing device 25).

The image processing device 25 is communicably connected to the control device 22, and the image processing device 25 performs image processing on the intestinal wall image 41 output from the control device 22. Details of the image processing in the image processing device 25 will be described below. The image processing device 25 outputs the intestinal wall image 41 subjected to the image processing to a predetermined output destination (for example, the display device 13). In addition, here, the form example in which the intestinal wall image 41 output from the control device 22 is output to the display device 13 through the image processing device 25 has been described. However, this is merely an example. The control device 22 and the display device 13 may be connected to each other, and the intestinal wall image 41 subjected to the image processing by the image processing device 25 may be displayed on the display device 13 through the control device 22.

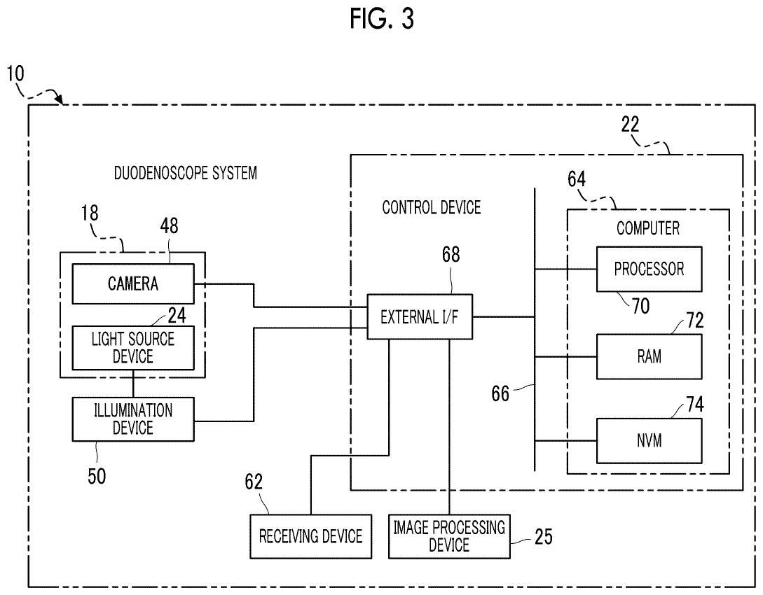

As shown in FIG. 3 as an example, the control device 22 comprises a computer 64, a bus 66, and an external I/F 68. The computer 64 comprises a processor 70, a RAM 72, and an NVM 74. The processor 70, the RAM 72, the NVM 74, and the external I/F 68 are connected to the bus 66.

For example, the processor 70 includes a CPU and a GPU and controls the entire control device 22. The GPU operates under the control of the CPU and is in charge of, for example, executing various processing operations of a graphics system and performing calculation using a neural network. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 includes a flash memory (for example, an EEPROM and/or an SSD). In addition, the flash memory is merely an example and may be other non-volatile storage devices, such as HDDs, or a combination of two or more types of non-volatile storage devices.

The external I/F 68 transmits and receives various types of information between a device (hereinafter, also referred to as an “external device”) outside the control device 22 and the processor 70. An example of the external I/F 68 is a USB interface.

The camera 48 is connected to the external I/F 68 as one of the external devices, and the external I/F 68 controls the exchange of various types of information between the camera 48 provided in the endoscope scope 18 and the processor 70. The processor 70 controls the camera 48 through the external I/F 68. In addition, the processor 70 acquires the intestinal wall image 41 (see FIG. 1) obtained by imaging the inside of the body of the subject 20 with the camera 48 provided in the endoscope scope 18 through the external I/F 68.

As one of the external devices, the light source device 24 is connected to the external I/F 68, and the external I/F 68 transmits and receives various types of information between the light source device 24 and the processor 70. The light source device 24 supplies light to the illumination device 50 under the control of the processor 70. The illumination device 50 performs irradiation with the light supplied from the light source device 24.

As one of the external devices, the receiving device 62 is connected to the external I/F 68. The processor 70 acquires the instruction received by the receiving device 62 through the external I/F 68 and executes the processing corresponding to the acquired instruction.

The image processing device 25 is connected to the external I/F 68 as one of the external devices, and the processor 70 outputs the intestinal wall image 41 to the image processing device 25 through the external I/F 68.

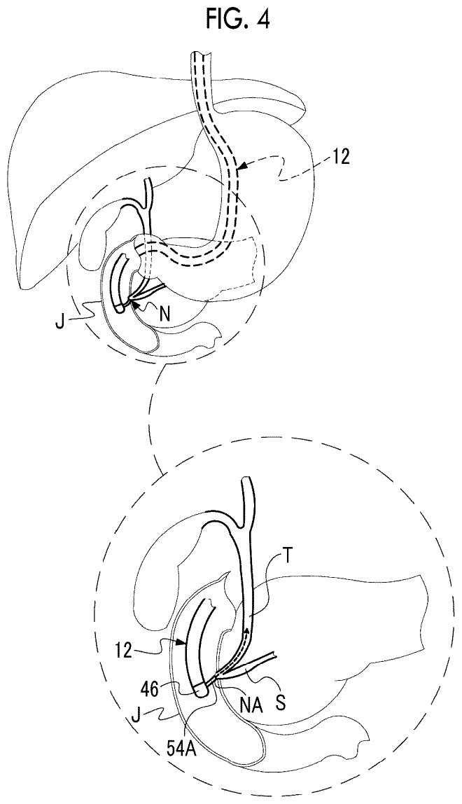

During the treatment on the duodenum using the endoscope, a treatment called endoscopic retrograde cholangio-pancreatography (ERCP) examination may be performed. As shown in FIG. 4 as an example, in the ERCP examination, for example, first, the duodenoscope 12 is inserted into a duodenum J through the esophagus and the stomach. In this case, the insertion state of the duodenoscope 12 may be checked by X-ray imaging. Then, the distal end part 46 of the duodenoscope 12 reaches the vicinity of a duodenal papilla N (hereinafter, also simply referred to as a “papilla N”) present in the intestinal wall of the duodenum J.

In the ERCP examination, for example, a cannula 54A is inserted from the papilla N. Here, the papilla N is a part that protrudes from the intestinal wall of the duodenum J, and an opening of an end part of a bile duct T (for example, a common bile duct, an intrahepatic bile duct, or a cystic duct) and a pancreatic duct S are present in a papillary protuberance NA of the papilla N. X-ray imaging is performed in a state in which a contrast agent is injected into the bile duct T, the pancreatic duct S, and the like through the cannula 54A from the opening of the papilla N. In this way, the ERCP examination includes various procedures such as the insertion of the duodenoscope 12 into the duodenum J, the checking of the position, orientation, and type of the papilla N, and the insertion of a treatment tool (for example, a cannula) into the papilla N. Therefore, the doctor 14 needs to operate the duodenoscope 12 and observe the state of the target part according to each procedure.

For example, in a case where the duodenoscope 12 is inserted into the duodenum J, in a state where the endoscope scope 18 of the duodenoscope 12 is inclined with respect to an intestinal tract direction, the papilla N is visually recognized in an inclined state. Therefore, there is a possibility that running directions of the bile duct T and the pancreatic duct S are erroneously recognized from the papilla N. Therefore, it is necessary to grasp to what extent the posture of the endoscope scope 18 is inclined with respect to the intestinal tract direction in the duodenum J.

Thus, in consideration of such circumstances, the medical support processing is performed by a processor 82 of the image processing device 25 in order to support the implementation of the medical care for the duodenum including the ERCP examination.

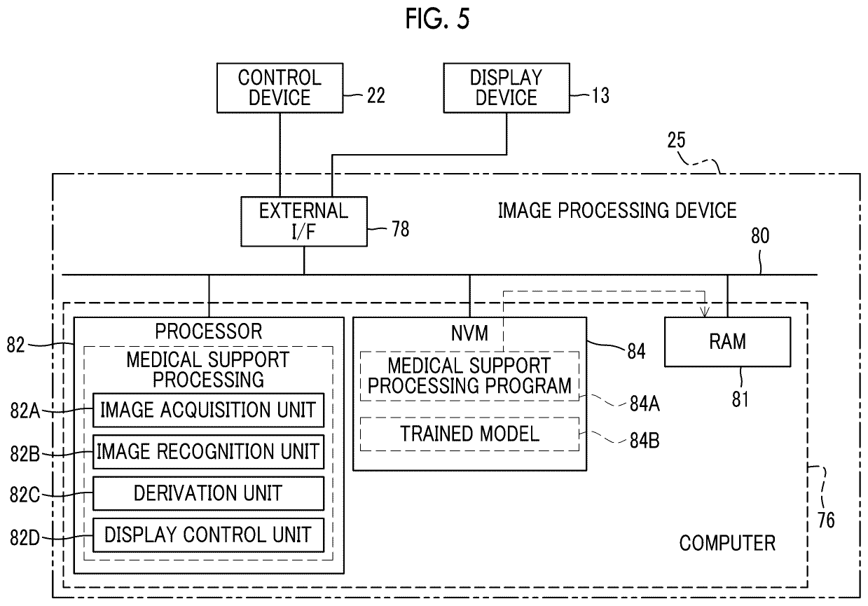

As shown in FIG. 5 as an example, the image processing device 25 comprises a computer 76, an external I/F 78, and a bus 80. The computer 76 comprises the processor 82, an NVM 84, and a RAM 81. The processor 82, the NVM 84, the RAM 81, and the external I/F 78 are connected to the bus 80. The computer 76 is an example of the “medical support device” and the “computer” according to the technology of the present disclosure. The processor 82 is an example of the “processor” according to the technology of the present disclosure.

In addition, a hardware configuration (that is, the processor 82, the NVM 84, and the RAM 81) of the computer 76 is essentially the same as a hardware configuration of the computer 64 shown in FIG. 3. Thus, the description of the hardware configuration of the computer 76 will be omitted here. In addition, since the role of the external I/F 78 in the image processing device 25 to transmit and receive information to and from the outside is essentially the same as the role performed by the external I/F 68 in the control device 22 shown in FIG. 3, the description thereof will be omitted here.

A medical support processing program 84A is stored in the NVM 84. The medical support processing program 84A is an example of the “program” according to the technology of the present disclosure. The processor 82 reads the medical support processing program 84A from the NVM 84 and executes the read medical support processing program 84A on the RAM 81. The medical support processing according to the present embodiment is realized by the processor 82 operating as an image acquisition unit 82A, an image recognition unit 82B, a derivation unit 82C, and a display control unit 82D in response to the medical support processing program 84A executed on the RAM 81.

A trained model 84B is stored in the NVM 84. In the present embodiment, the image recognition unit 82B performs image recognition processing using an AI method as the image recognition processing for object detection. The trained model 84B is optimized by performing machine learning on the neural network in advance.

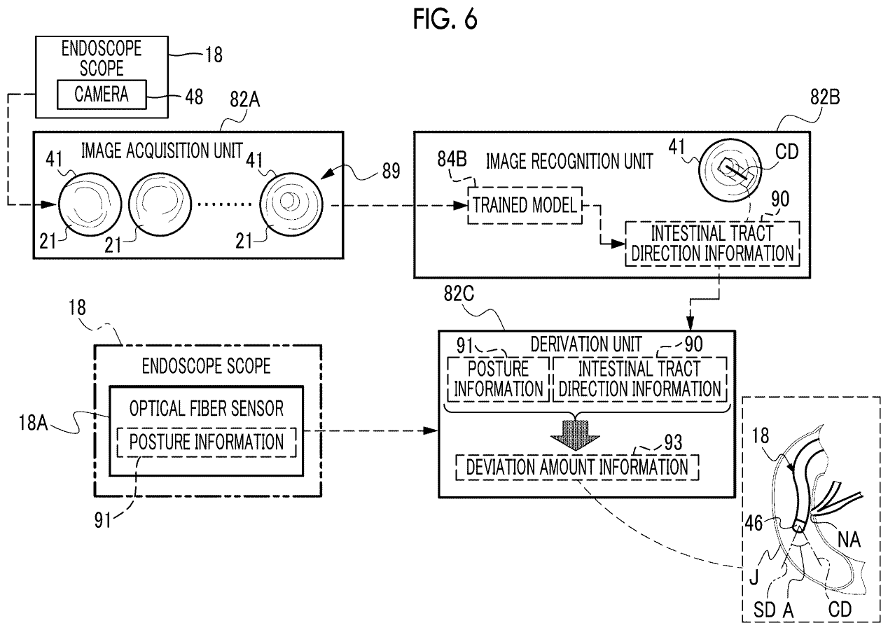

As shown in FIG. 6 as an example, the image acquisition unit 82A acquires the intestinal wall images 41, which have been generated by the camera 48 capturing the images at an imaging frame rate (for example, several tens of frames/sec), from the camera 48 in units of one frame.

The image acquisition unit 82A holds a time-series image group 89. The time-series image group 89 is a plurality of time-series intestinal wall images 41 in which the observation target 21 is captured. The time-series image group 89 includes, for example, a predetermined number of frames (for example, a predetermined number of frames within a range of several tens to several hundreds of frames) of intestinal wall images 41. The image acquisition unit 82A updates a time-series image group 89 using a FIFO method each time the intestinal wall image 41 is acquired from the camera 48.

Here, the form example in which the time-series image group 89 is held and updated by the image acquisition unit 82A has been described, but this is merely an example. For example, the time-series image group 89 may be held and updated in a memory, such as the RAM 81, which is connected to the processor 82.

The image recognition unit 82B performs image recognition processing using the trained model 84B on the time-series image group 89. By performing the image recognition processing, an intestinal tract direction CD included in the observation target 21 is detected. Here, the intestinal tract direction CD refers to a luminal direction of the duodenum. Here, the detection of the intestinal tract direction refers to the processing of storing intestinal tract direction information 90 (for example, position coordinates indicating the direction in which the duodenum extends) that is information capable of specifying the intestinal tract direction CD and the intestinal wall image 41 in the memory in an associated state. The intestinal tract direction information 90 is an example of the “intestinal tract direction-related information” according to the technology of the present disclosure.

The trained model 84B is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the intestinal tract direction CD.

Here, an example of the annotation in the correct answer data includes an annotation (for example, an annotation in which a line segment connecting centers of arc shapes of the fold shapes is set as the intestinal tract direction CD) of the intestinal tract direction CD based on the fold shape of the intestinal tract shown in the intestinal wall image 41. In addition, in a case where the intestinal wall image 41 is a depth image, examples of the annotation (for example, an annotation in which a direction in which depth in a depth direction indicated by depth information increases is set as the intestinal tract direction CD) in the other correct answer data include an annotation based on the depth information.

In addition, here, a form in which only one trained model 84B is used by the image recognition unit 82B is given as an example, but this is merely an example. For example, the trained model 84B selected from a plurality of the trained models 84B may be used by the image recognition unit 82B. In this case, each trained model 84B is created by performing machine learning specialized for each procedure (for example, the position of the duodenoscope 12 with respect to the papilla N, or the like) of the ERCP examination, and the trained model 84B corresponding to the procedure of the ERCP examination currently being performed may be selected and used by the image recognition unit 82B.

The image recognition unit 82B inputs the intestinal wall image 41 acquired from the image acquisition unit 82A to the trained model 84B. Accordingly, the trained model 84B outputs the intestinal tract direction information 90 corresponding to the input intestinal wall image 41. The image recognition unit 82B acquires the intestinal tract direction information 90 output from the trained model 84B.

The derivation unit 82C derives the amount of deviation (hereinafter, simply referred to as a “deviation amount”) of the endoscope scope 18 with respect to the intestinal tract direction CD. Here, the deviation amount refers to the degree of deviation between the posture of the endoscope scope 18 and the intestinal tract direction CD. Specifically, the deviation amount refers to the deviation amount between a direction along an imaging surface of an imaging element of the camera 48 provided in the endoscope scope 18 (for example, an up-down direction in the angle of view) and the intestinal tract direction CD. In addition, since the camera 48 is provided at the distal end part 46, the deviation amount can also be said to be an angle between a longitudinal direction SD (for example, a central axis direction in a case where the distal end part 46 has a cylindrical shape) of the distal end part 46 and the intestinal tract direction CD.

The derivation unit 82C acquires the intestinal tract direction information 90 from the image recognition unit 82B. In addition, the derivation unit 82C acquires posture information 91 from an optical fiber sensor 18A provided in the endoscope scope 18. The posture information 91 is information indicating the posture of the endoscope scope 18. The optical fiber sensor 18A is a sensor disposed inside the endoscope scope 18 (for example, the insertion part 44 and the distal end part 46) in the longitudinal direction. By using the optical fiber sensor 18A, the posture (for example, the inclination of the distal end part 46 from a reference position (for example, a straight state of the endoscope scope 18)) of the endoscope scope 18 can be detected. In this case, for example, a known endoscope posture detection technology of JP6797834B or the like can be appropriately used. The posture information 91 is an example of the “posture information” according to the technology of the present disclosure.

In addition, here, the posture detection technology using the optical fiber sensor 18A has been described, but this is merely an example. For example, the inclination of the distal end part 46 of the endoscope scope 18 may be detected by using a so-called electromagnetic navigation method. In this case, for example, a known endoscope posture detection technology of JP6534193B or the like can be appropriately used.

The derivation unit 82C derives deviation amount information 93 that is information indicating the deviation amount, by using the intestinal tract direction information 90 and the posture information 91. In the example shown in FIG. 6, an angle A is shown as the deviation amount information 93. The derivation unit 82C derives the deviation amount using, for example, a deviation amount calculation expression (not shown). The deviation amount calculation expression is a calculation expression in which the position coordinates of the intestinal tract direction CD indicated by the intestinal tract direction information 90 and the position coordinates of the distal end part 46 in the longitudinal direction SD indicated by the posture information 91 are set as independent variables, and the angle between the intestinal tract direction CD and the longitudinal direction SD of the distal end part 46 is set as a dependent variable. The deviation amount information 93 is an example of the “deviation amount information” according to the technology of the present disclosure.

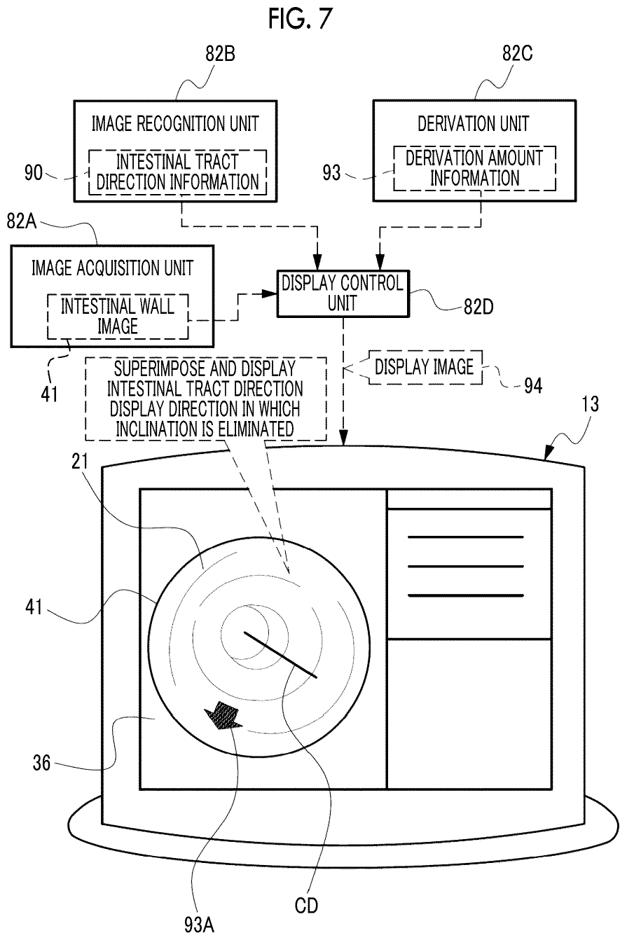

As shown in FIG. 7 as an example, the display control unit 82D acquires the intestinal wall image 41 from the image acquisition unit 82A. In addition, the display control unit 82D acquires the intestinal tract direction information 90 from the image recognition unit 82B. Moreover, the display control unit 82D acquires the deviation amount information 93 from the derivation unit 82C. The display control unit 82D generates an operation instruction image 93A for matching the longitudinal direction SD of the distal end part 46 with the intestinal tract direction CD, according to the deviation amount indicated by the deviation amount information 93. The operation instruction image 93A is, for example, an arrow indicating an operation direction of the distal end part 46 in which the deviation amount is reduced. The display control unit 82D generates a display image 94 including the intestinal wall image 41, the intestinal tract direction CD indicated by the intestinal tract direction information 90, and the operation instruction image 93A, and outputs the display image 94 to the display device 13. Specifically, the display control unit 82D performs graphical user interface (GUI) control for displaying the display image 94 to cause the screen 36 to be displayed on the display device 13. The screen 36 is an example of the “first screen” according to the technology of the present disclosure. The operation instruction image 93A is an example of “posture adjustment support information” according to the technology of the present disclosure.

In addition, here, the form example in which the operation instruction image 93A is displayed on the screen 36 to allow a user to grasp the deviation amount has been described, but the technology of the present disclosure is not limited to this. For example, a message (not shown) indicating the operation content for reducing the deviation amount may be displayed on the screen 36. An example of the message is “Please incline the distal end part of the duodenoscope toward the back side by 10 degrees.” A voice output device such as a speaker may notify the user.

The user can grasp the intestinal tract direction CD by visually recognizing the screen 36 of the display device 13. In addition, by visually recognizing the operation instruction image 93A displayed on the screen 36, it is possible to grasp the operation for reducing the deviation between the distal end part 46 of the endoscope scope 18 and the intestinal tract direction CD.

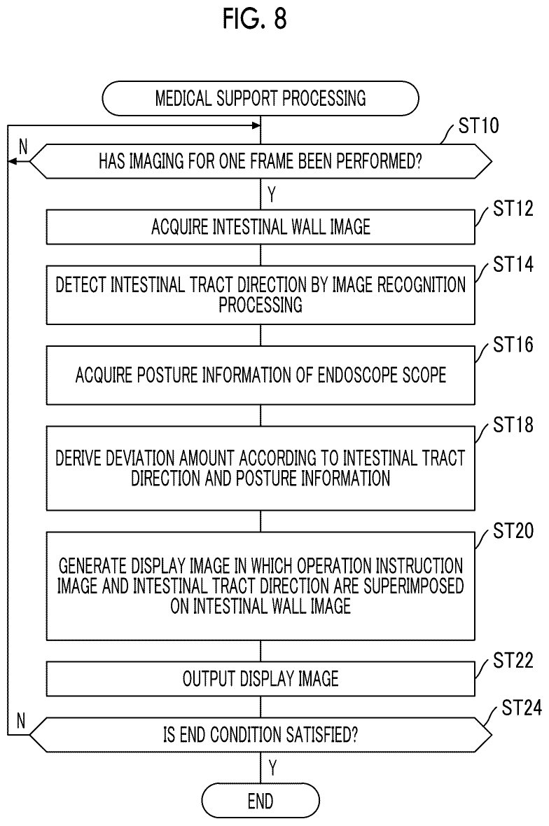

Next, the operation of a portion of the duodenoscope system 10 according to the technology of the present disclosure will be described with reference to FIG. 8.

FIG. 8 shows an example of a flow of the medical support processing performed by the processor 82. The flow of the medical support processing shown in FIG. 8 is an example of the “medical support method” according to the technology of the present disclosure.

In the medical support processing shown in FIG. 8, first, in step ST10, the image acquisition unit 82A determines whether or not imaging for one frame has been performed by the camera 48 provided in the endoscope scope 18. In a case where the imaging for one frame has not been performed by the camera 48 in step ST10, the determination result is “No”, and the determination in step ST10 is performed again. In a case where the imaging for one frame has been performed by the camera 48 in step ST10, the determination result is “Yes”, and the medical support processing proceeds to step ST12.

In step ST12, the image acquisition unit 82A acquires one frame of the intestinal wall image 41 from the camera 48 provided in the endoscope scope 18. After the processing in step ST12 is executed, the medical support processing proceeds to step ST14.

In step ST14, the image recognition unit 82B performs image recognition processing (that is, image recognition processing using the trained model 84B) using the AI method on the intestinal wall image 41 acquired in step ST12 to detect the intestinal tract direction CD. After the processing in step ST14 is executed, the medical support processing proceeds to step ST16.

In step ST16, the derivation unit 82C acquires the posture information 91 from the optical fiber sensor 18A of the endoscope scope 18. After the processing in step ST16 is executed, the medical support processing proceeds to step ST18.

In step ST18, the derivation unit 82C derives the deviation amount based on the intestinal tract direction CD obtained by the image recognition unit 82B in step ST14 and the posture information 91 acquired in step ST16. Specifically, the derivation unit 82C derives an angle between the intestinal tract direction CD and the longitudinal direction SD of the distal end part 46 indicated by the posture information 91. After the processing in step ST18 is executed, the medical support processing proceeds to step ST20.

In step ST20, the display control unit 82D generates the display image 94 on which the operation instruction image 93A and the intestinal tract direction CD according to the deviation amount derived in step ST18 are superimposed and displayed on the intestinal wall image 41. After the processing in step ST20 is executed, the medical support processing proceeds to step ST22.

In step ST22, the display control unit 82D outputs the display image 94 generated in step ST20 to the display device 13. After the processing in step ST22 is executed, the medical support processing proceeds to step ST24.

In step ST24, the display control unit 82D determines whether or not a condition for ending the medical support processing is satisfied. An example of the medical support processing end condition is a condition (for example, a condition in which an instruction to end the medical support processing is received by the receiving device 62) in which an instruction to end the medical support processing is issued to the duodenoscope system 10.

In a case where a condition to end the medical support processing is not satisfied in step ST24, the determination result is “No”, and the medical support processing proceeds to step ST10. In a case where the condition to end the medical support processing is satisfied in step ST24, the determination result is “Yes”, and the medical support processing ends.

As described above, in the duodenoscope system 10 according to the present first embodiment, the image recognition processing is performed on the intestinal wall image 41 in the image recognition unit 82B of the processor 82, and the intestinal tract direction CD in the intestinal wall image 41 is detected as a result of the image recognition processing. Then, the intestinal tract direction information 90 indicating the intestinal tract direction CD is output to the display control unit 82D, and the display image 94 generated in the display control unit 82D is output to the display device 13. The display image 94 includes the intestinal tract direction CD superimposed and displayed on the intestinal wall image 41. Accordingly, the user can recognize the intestinal tract direction CD. According to the present configuration, it is possible to easily allow the user to grasp to what extent the posture of the endoscope scope 18 deviates with respect to the intestinal tract direction CD.

In addition, in the duodenoscope system 10 according to the present first embodiment, the deviation amount information 93 is derived in the derivation unit 82C. The deviation amount information 93 indicates a deviation amount between the posture of the endoscope scope 18 and the intestinal tract direction CD. The deviation amount information 93 is output to the display control unit 82D, and the display image 94 generated by the display control unit 82D is output to the display device 13. The display image 94 includes a display based on the deviation amount information 93. Accordingly, the user can recognize the deviation amount between the posture of the endoscope scope 18 and the intestinal tract direction CD. According to the present configuration, it is possible to easily allow the user to grasp to what extent the posture of the endoscope scope 18 deviates with respect to the intestinal tract direction CD.

In addition, in the duodenoscope system 10 according to the present first embodiment, the image recognition processing is performed on the intestinal wall image 41 in the image recognition unit 82B, so that the intestinal tract direction information 90 indicating the intestinal tract direction CD is obtained. Accordingly, the intestinal tract direction information 90 with higher accuracy is obtained compared to a case where the user designates the intestinal tract direction CD with respect to the intestinal wall image 41 by visual observation.

In addition, in the duodenoscope system 10 according to the present first embodiment, the intestinal tract direction information 90 is output to the display device 13 by the display control unit 82D, and the intestinal tract direction CD is displayed on the screen 36 of the display device 13. Accordingly, it is possible to allow the user to easily visually grasp to what extent the posture of the endoscope scope 18 deviates with respect to the intestinal tract direction CD.

In addition, in the duodenoscope system 10 according to the present first embodiment, the derivation unit 82C acquires the posture information 91, which is information capable of specifying the posture of the endoscope scope 18, from the optical fiber sensor 18A. In addition, in the derivation unit 82C, the deviation amount information 93 is generated based on the posture information 91 and the intestinal tract direction information 90. Moreover, in the display control unit 82D, the operation instruction image 93A indicating the operation direction in which the deviation amount is reduced is generated based on the deviation amount information 93. The display control unit 82D outputs the operation instruction image 93A to the display device 13, and the operation instruction image 93A is superimposed and displayed on the intestinal wall image 41 on the display device 13. Accordingly, in a state in which the endoscope scope 18 is inserted into the duodenum, it is easy for the user to set the posture of the endoscope scope 18 with respect to the intestinal tract direction CD to an intended posture. For example, the user can bring the intestinal tract direction CD and the posture of the endoscope scope 18 closer to each other by performing the operation of changing the posture of the endoscope scope 18 in the direction indicated by the operation instruction image 93A.

In the above first embodiment, the form example in which the intestinal tract direction CD is detected by the image recognition processing using the AI method has been described, but the technology of the present disclosure is not limited to this. For example, the intestinal tract direction CD may be detected by image recognition processing using a pattern matching method. In this case, for example, a form may be adopted in which a region (that is, a fold region) indicating a fold of the intestinal tract included in the intestinal wall image 41 is detected, and the intestinal tract direction is estimated from the arc shape of the fold region (for example, a line connecting the centers of the arcs is estimated as the intestinal tract direction).

### First Modification Example

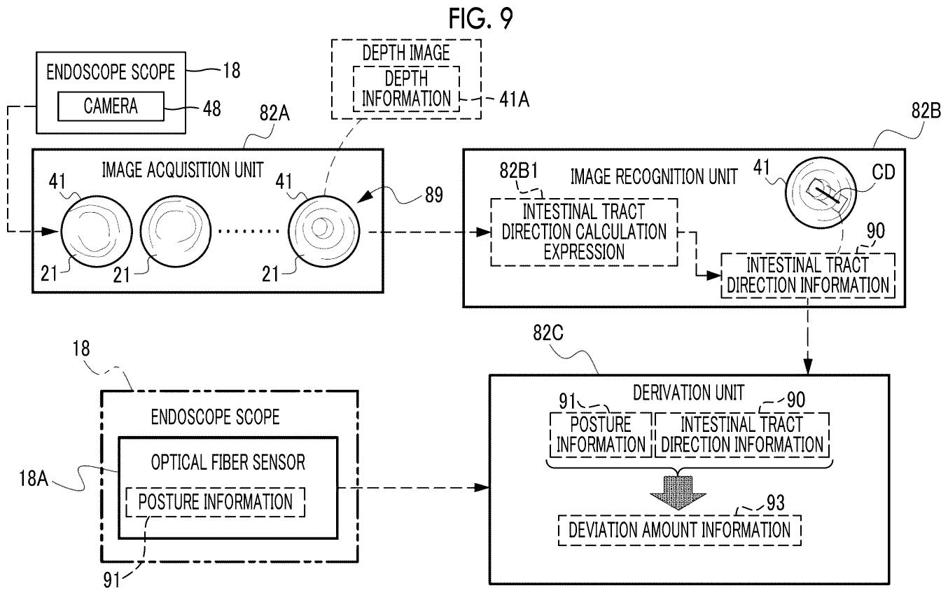

In the above first embodiment, the form example in which the detection of the intestinal tract direction CD is performed using the intestinal wall image 41 that does not include the depth information has been described, but the technology of the present disclosure is not limited to this. In the present first modification example, the image recognition unit 82B derives the intestinal tract direction using the intestinal wall image 41 which is the depth image. As shown in FIG. 9 as an example, the intestinal wall image 41 is a depth image having depth information 41A that is information indicating the depth (that is, the distance to the intestinal wall) of the duodenum, which is a subject, as a pixel value. The depth of the duodenum is obtained by, for example, distance measurement using a so-called ToF method by a distance-measuring sensor mounted on the distal end part 46. The image recognition unit 82B acquires the intestinal wall image 41 from the image acquisition unit 82A. The depth information 41A is an example of the “depth information” according to the technology of the present disclosure.

The image recognition unit 82B derives the intestinal tract direction information 90 based on the depth information 41A indicated by the intestinal wall image 41. The image recognition unit 82B derives the intestinal tract direction information 90, for example, by using an intestinal tract direction calculation expression 82B1. The intestinal tract direction calculation expression 82B1 is, for example, a calculation expression in which the depth indicated by the depth information 41A is set as an independent variable and a position coordinate group of an axis line indicating the intestinal tract direction CD is set as a dependent variable. In this way, the intestinal tract direction information 90 is obtained based on the depth information 41A of the intestinal wall image 41.

As described above, in the duodenoscope system 10 according to the present first modification example, the intestinal wall image 41 has the depth information 41A indicating the depth of the duodenum, and the intestinal tract direction information 90 is acquired based on the depth information 41A. The intestinal tract direction CD is a direction along a depth direction in a lumen of the duodenum. The depth information 41A reflects the depth of the lumen of the duodenum. Therefore, since the intestinal tract direction CD is derived based on the depth information 41A, the intestinal tract direction information 90 indicating the intestinal tract direction CD with higher accuracy is obtained compared to a case where the depth information 41A is not considered.

### Second Modification Example

In the above first embodiment, the form example in which the intestinal tract direction CD is obtained by the image recognition processing on the intestinal wall image 41 has been described, but the technology of the present disclosure is not limited to this. In the present second modification example, a direction (hereinafter, also simply referred to as a “predetermined direction”) intersecting the intestinal tract direction CD at a predetermined angle is obtained.

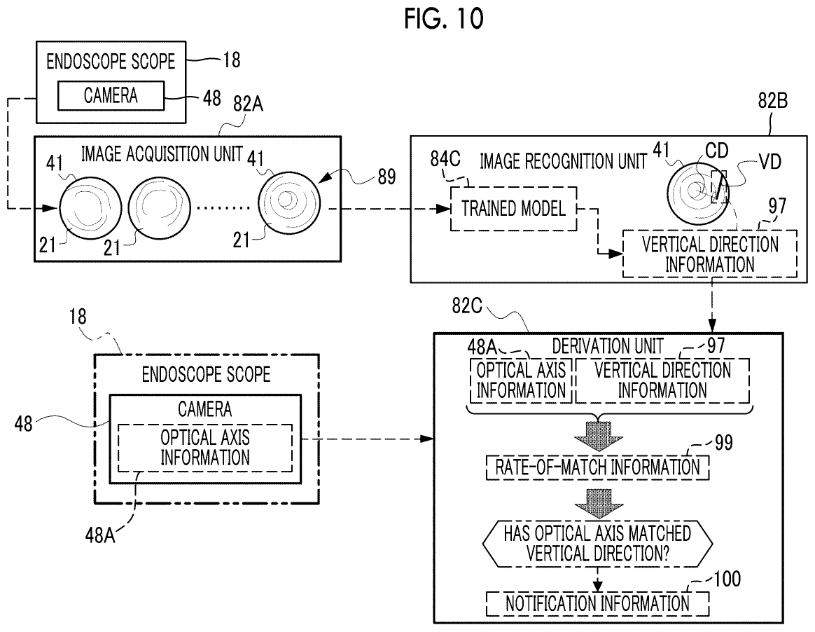

As shown in FIG. 10 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to the trained model 84C. Accordingly, the trained model 84C outputs vertical direction information 97 corresponding to the input time-series image group 89. The image recognition unit 82B acquires the vertical direction information 97 output from the trained model 84C. Here, the vertical direction information 97 is information (for example, a position coordinate group indicating an axis line perpendicular to the intestinal tract direction CD) capable of specifying a direction VD (hereinafter, also simply referred to as a “vertical direction VD”) perpendicular to the intestinal tract direction CD.

In the image recognition processing using the trained model 84C, the degree of certainty of the identification result is calculated according to the result of specifying the direction perpendicular to the intestinal tract direction CD. Here, the degree of certainty is a statistical measure indicating the certainty of the identification result. The degree of certainty is, for example, a score input to an activation function (for example, a softmax function or the like) of an output layer of the trained model 84C. The vertical direction information 97 output from the trained model 84C has a score equal to or greater than a threshold value (for example, equal to or greater than 0.9).

In addition, in the present embodiment, “vertical” indicates vertical in a meaning including an error that is generally allowed in the technical field to which the technology of the present disclosure belongs, and an error to such an extent not contrary to the gist of the technology of the present disclosure, in addition to completely vertical. In addition, here, the vertical direction with respect to the intestinal tract direction CD is exemplified as the predetermined angle with respect to the intestinal tract direction CD, but the technology of the present disclosure is not limited to this. For example, the predetermined angle may be 45 degrees, 60 degrees, or 80 degrees.

The trained model 84C is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the vertical direction VD.

The derivation unit 82C derives the rate of match between the predetermined direction and the direction of an optical axis of the camera 48. The fact that the predetermined direction and the direction of the optical axis match means that a direction in which the camera 48 is directed matches a direction predetermined by the user. That is, this means that the distal end part 46 provided with the camera 48 is not in a direction (for example, a direction inclined with respect to the intestinal tract direction CD) that is not intended by the user.

Thus, the derivation unit 82C acquires the vertical direction information 97. In addition, the derivation unit 82C acquires optical axis information 48A from the camera 48 of the endoscope scope 18. The optical axis information 48A is information for specifying an optical axis of an optical system of the camera 48. Then, the derivation unit 82C generates rate-of-match information 99 by comparing a direction indicated by the vertical direction information 97 with the direction of the optical axis indicated by the optical axis information 48A. The rate-of-match information 99 is information indicating the rate of match (for example, an angle formed between the direction of the optical axis and the predetermined direction) between the direction of the optical axis and the predetermined direction. In addition, in the present embodiment, “match” refers to a match in the sense of including an error generally allowed in the technical field to which the technology of the present disclosure belongs, that is, an error to the extent that it does not contradict the gist of the technology of the present disclosure, in addition to an exact match. The vertical direction information 97 is an example of the “first direction information” and the “intestinal tract direction-related information” according to the technology of the present disclosure.

Moreover, the derivation unit 82C determines whether or not the direction of the optical axis matches the predetermined direction. In a case where the direction of the optical axis matches the predetermined direction, the derivation unit 82C generates notification information 100. The notification information 100 is information for notifying the user that the direction of the optical axis matches the predetermined direction (for example, text indicating that the direction of the optical axis matches the predetermined direction).

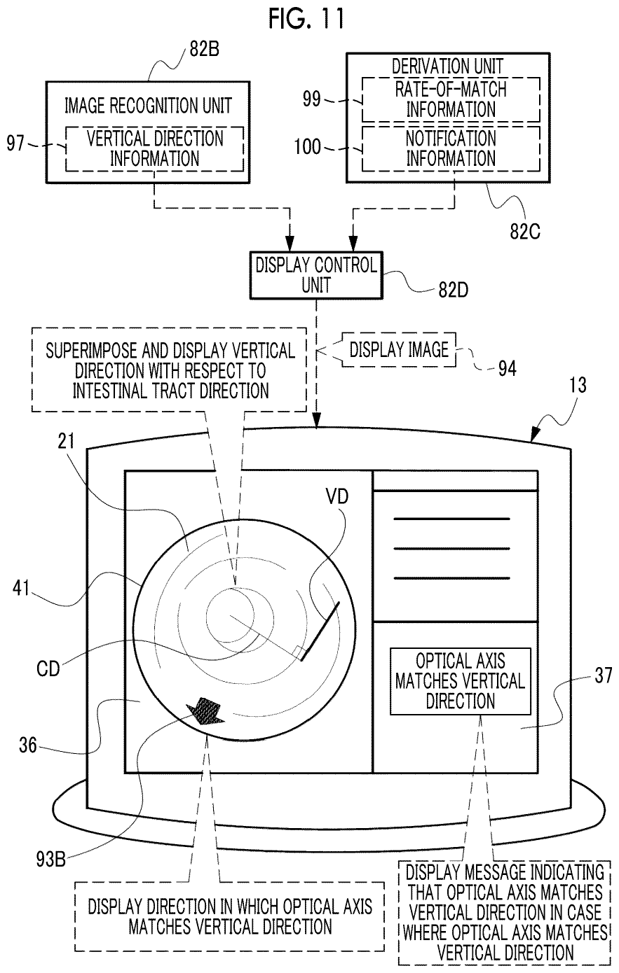

As shown in FIG. 11 as an example, the display control unit 82D acquires the vertical direction information 97 from the image recognition unit 82B. In addition, the display control unit 82D acquires the rate-of-match information 99 from the derivation unit 82C. The display control unit 82D generates an operation instruction image 93B (for example, an arrow indicating an operation direction) for matching the direction of the optical axis with the predetermined direction according to the rate of match between the direction of the optical axis indicated by the rate-of-match information 99 and the predetermined direction. Then, the display control unit 82D generates a display image 94 including the vertical direction VD indicated by the vertical direction information 97, the operation instruction image 93B, and the intestinal wall image 41, and outputs the display image 94 to the display device 13. In the example shown in FIG. 11, the display device 13 shows the intestinal wall image 41 on which the vertical direction VD and the operation instruction image 93B are superimposed and displayed on the screen 36. The vertical direction VD is an example of the “first direction”, the “second direction”, and the “third direction” according to the technology of the present disclosure. The operation instruction image 93B is an example of the “condition information” according to the technology of the present disclosure.

In addition, in a case where the direction of the optical axis matches the predetermined direction, the derivation unit 82C outputs the notification information 100 to the display control unit 82D instead of the rate-of-match information 99. In this case, the display control unit 82D generates the display image 94 including the content for notifying the user that the direction of the optical axis indicated by the notification information 100 matches the predetermined direction, instead of the operation instruction image 93B. The example shown in FIG. 11 shows that, on the display device 13, an example in which a message “the optical axis matches the vertical direction” is displayed on the screen 37. The notification information 100 is an example of the “notification information” according to the technology of the present disclosure.

In addition, here, the form example in which a message based on the notification information 100 is displayed on the display device 13 has been described, but this is merely an example. For example, a symbol such as a circle mark based on the notification information 100 may be displayed. In addition, the notification information 100 may be output to a voice output device such as a speaker instead of the display device 13 or together with the display device 13.

As described above, in the duodenoscope system 10 according to the present second modification example, the derivation unit 82C derives the vertical direction information 97 that is information capable of specifying the direction perpendicular to the intestinal tract direction CD. The vertical direction information 97 is output to the display control unit 82D, and the display image 94 generated by the display control unit 82D is output to the display device 13. The display image 94 includes the vertical direction VD indicated by the vertical direction information 97. Accordingly, the user can recognize the direction intersecting the intestinal tract direction CD at the predetermined angle.

In addition, in the duodenoscope system 10 according to the present second modification example, the image recognition processing is performed on the intestinal wall image 41 in the image recognition unit 82B, so that the vertical direction information 97 indicating the vertical direction VD is obtained. Accordingly, the vertical direction information 97 with high accuracy is obtained compared to a case where the user designates the vertical direction VD with respect to the intestinal wall image 41 by visual observation.

In addition, in the duodenoscope system 10 according to the present second modification example, in the image recognition processing using the trained model 84C in the image recognition unit 82B, the vertical direction information 97 is obtained with a degree of certainty equal to or greater than a threshold value. Accordingly, in the image recognition processing using the trained model 84C in the image recognition unit 82B, the vertical direction information 97 with higher accuracy is obtained compared to a case where the threshold value is not set for the degree of certainty.

In addition, in the duodenoscope system 10 according to the present second modification example, the derivation unit 82C acquires the optical axis information 48A from the camera 48. In addition, in the derivation unit 82C, the rate-of-match information 99 is generated based on the optical axis information 48A and the vertical direction information 97. Moreover, in the display control unit 82D, the display image 94 is generated based on the rate-of-match information 99 and is output to the display device 13. The display image 94 includes a display related to the rate of match between the direction of the optical axis indicated by the rate-of-match information 99 and the predetermined direction. Accordingly, the user can grasp to what extent the optical axis of the camera 48 deviates from the vertical direction VD. For example, in a case where the optical axis matches the vertical direction VD, there is a high probability that the camera 48 faces the intestinal wall of the duodenum. By maintaining the posture of the endoscope scope 18 in this state, it is easy to find the papilla N present in the intestinal wall of the duodenum, and it is also easy to cause the camera 48 to directly face the papilla N.

In addition, in the duodenoscope system 10 according to the present second modification example, the display control unit 82D generates the operation instruction image 93B for matching the direction of the optical axis with the predetermined direction based on the rate-of-match information 99. The display control unit 82D outputs the operation instruction image 93B to the display device 13, and the operation instruction image 93B is superimposed and displayed on the intestinal wall image 41 on the display device 13. Accordingly, the user can grasp the operation required to match the optical axis direction of the camera 48 with the vertical direction VD.

In addition, in the duodenoscope system 10 according to the present second modification example, the derivation unit 82C determines whether or not the direction of the optical axis matches the predetermined direction. In a case where the direction of the optical axis matches the predetermined direction, the derivation unit 82C generates the notification information 100. In the display control unit 82D, the display image 94 is generated based on the notification information 100 and is output to the display device 13. The display image 94 includes a display indicating that the direction of the optical axis indicated by the notification information 100 matches the predetermined direction. Accordingly, the user can be made to perceive that the direction of the optical axis matches the predetermined direction.

### Third Modification Example

In the above first embodiment, the form example in which the intestinal tract direction CD is obtained by the image recognition processing on the intestinal wall image 41 has been described, but the technology of the present disclosure is not limited to this. In the present third modification example, a running direction TD of the bile duct is obtained based on the intestinal tract direction CD.

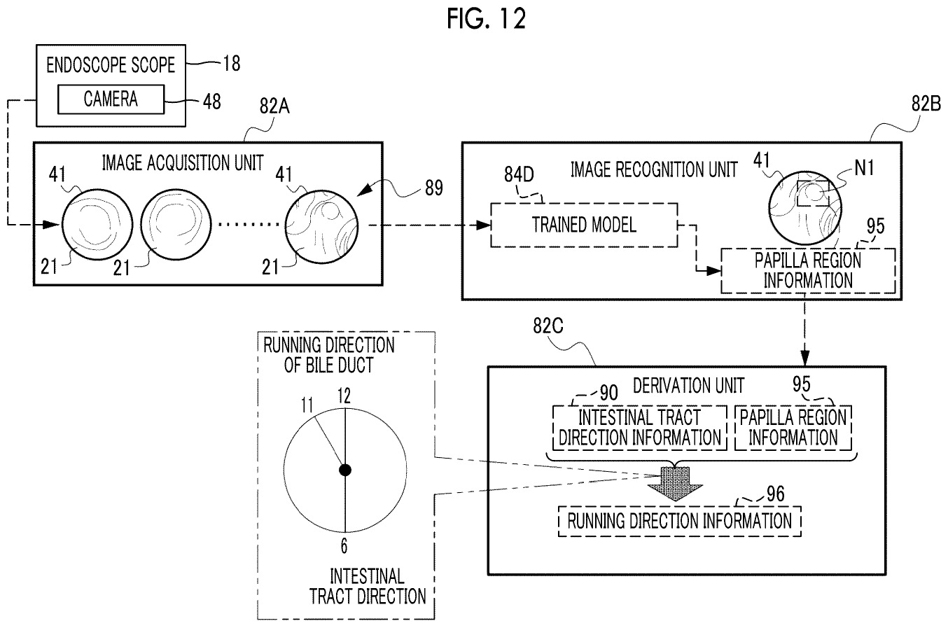

As shown in FIG. 12 as an example, the image acquisition unit 82A updates the time-series image group 89 using the FIFO method each time the intestinal wall image 41 is acquired from the camera 48.

The image recognition unit 82B performs papilla detection processing using a trained model 84D on the time-series image group 89. The image recognition unit 82B acquires the time-series image group 89 from the image acquisition unit 82A and inputs the acquired time-series image group 89 to the trained model 84D. Accordingly, the trained model 84D outputs papilla region information 95 corresponding to the input time-series image group 89. The image recognition unit 82B acquires the papilla region information 95 output from the trained model 84D. Here, the papilla region information 95 includes information (for example, coordinates and a range in the image) for specifying a papilla region N1 in the intestinal wall image 41 in which the papilla N is captured.

The trained model 84D is obtained by performing machine learning using training data on the neural network to optimize the neural network. The training data is a plurality of pieces of data (that is, a plurality of frames of data) in which example data and correct answer data are associated with each other. The training data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the papilla region N1.

The derivation unit 82C derives running direction information 96 that is information indicating the running direction TD of the bile duct. The running direction information 96 includes information (for example, position coordinates indicating the direction in which the bile duct extends) capable of specifying the direction in which the bile duct extends. The derivation unit 82C acquires the papilla region information 95 from the image recognition unit 82B. In addition, the derivation unit 82C acquires the intestinal tract direction information 90 obtained by the image recognition processing using the trained model 84B (see FIG. 6) from the image recognition unit 82B. Then, the derivation unit 82C derives the running direction information 96 based on the intestinal tract direction information 90 and the papilla region information 95. The derivation unit 82C derives the running direction TD from, for example, a predetermined orientation relationship between the intestinal tract direction CD and the running direction TD. Specifically, the derivation unit 82C derives the running direction TD as a direction of 11 o'clock to 12 o'clock in a case where the intestinal tract direction CD is a direction of 6 o'clock. Moreover, the derivation unit 82C uses the papilla region N1 indicated by the papilla region information 95 as a starting point in the running direction TD.

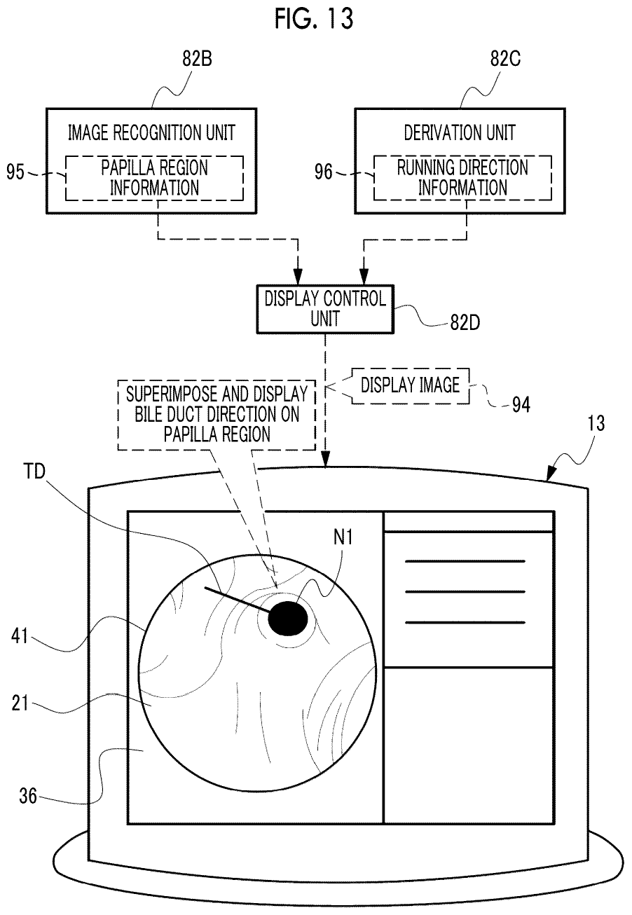

As shown in FIG. 13 as an example, the display control unit 82D acquires the running direction information 96 from the derivation unit 82C. In addition, the display control unit 82D acquires the papilla region information 95 from the image recognition unit 82B. The display control unit 82D generates the display image 94 on which the running direction TD indicated by the running direction information 96 and the papilla region N1 indicated by the papilla region information 95 are superimposed and displayed on the intestinal wall image 41 acquired from the image acquisition unit 82A (see FIG. 6), and outputs the display image 94 to the display device 13. On the display device 13, the intestinal wall image 41 on which the running direction TD is superimposed and displayed is displayed on the screen 36.

As described above, in the duodenoscope system 10 according to the present third modification example, the image recognition unit 82B performs the papilla detection processing using the trained model 84D. The papilla region information 95 is obtained by the papilla detection processing. In addition, the image recognition unit 82B performs the image recognition processing using the trained model 84A to obtain the intestinal tract direction information 90. The derivation unit 82C derives the running direction information 96 based on the intestinal tract direction information 90 and the papilla region information 95. Then, the display image 94 is output to the display device 13 by the display control unit 82D. The display image 94 includes the papilla region N1 indicated by the papilla region information 95 and the running direction TD of the bile duct indicated by the running direction information 96. On the display device 13, the papilla region N1 and the running direction TD of the bile duct are displayed on the screen 36. Accordingly, it is possible to make it easier for the user who observes the papilla N through the screen 36 to visually grasp the running direction TD of the bile duct.

For example, in the ERCP examination, the camera 48 may be made to directly face the papilla N. In this case, by using the running direction of the bile duct or the pancreatic duct, it is easy to grasp the posture of the endoscope scope 18. In addition, in a case where a treatment tool is inserted into the papilla N, the running direction of the bile duct or the pancreatic duct is grasped, so that it is easy to perform the operation of inserting a tube into the bile duct or the pancreatic duct in the papilla N.

### Fourth Modification Example

In the above first embodiment, the form example in which the intestinal tract direction CD is obtained by the image recognition processing on the intestinal wall image 41 has been described, but the technology of the present disclosure is not limited to this. In the present fourth modification example, the orientation of the papillary protuberance NA in the papilla N (hereinafter, also simply referred to as a “papilla orientation ND”) is obtained based on the intestinal tract direction CD.

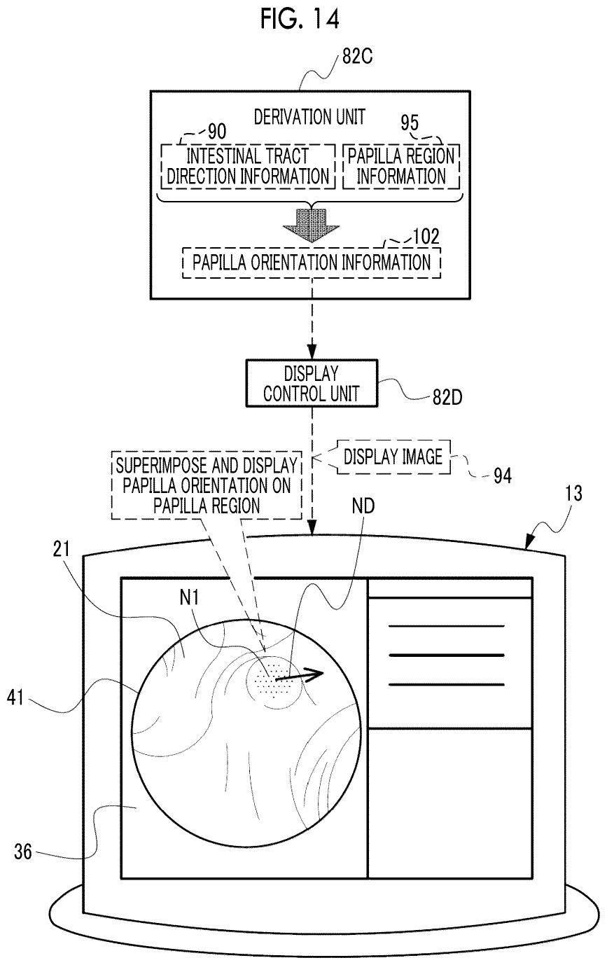

The image recognition unit 82B performs the image recognition processing on the intestinal wall image 41 to obtain the intestinal tract direction information 90 and the papilla region information 95 (see FIG. 12). As an example, as shown in FIG. 14, the derivation unit 82C generates papilla orientation information 102 based on the intestinal tract direction information 90 and the papilla region information 95. The papilla orientation information 102 is information capable of specifying the papilla orientation ND (for example, an orientation in which the papillary protuberance NA faces the treatment tool). The papilla orientation ND is obtained, for example, as a tangent line at the papillary protuberance NA in the running direction TD of the bile duct. Thus, the derivation unit 82C derives the running direction TD of the bile duct from the intestinal tract direction CD indicated by the intestinal tract direction information 90, and further derives the direction of the tangent line at the papillary protuberance NA from the running direction TD as the papilla orientation ND.

The display control unit 82D acquires the papilla orientation information 102 from the derivation unit 82C. The display control unit 82D generates the display image 94 on which the papilla orientation ND indicated by the papilla orientation information 102 and the papilla region N1 indicated by the papilla region information 95 are superimposed and displayed on the intestinal wall image 41 acquired from the image acquisition unit 82A (see FIG. 6), and outputs the display image 94 to the display device 13. On the display device 13, the intestinal wall image 41 on which the papilla orientation ND is superimposed and displayed is displayed on the screen 36.

Here, the form example in which the papilla orientation ND is displayed as an arrow has been described, but this is merely an example. The papilla orientation ND may be an aspect in which a direction is indicated by text.

As described above, in the duodenoscope system 10 according to the present fourth modification example, the papilla detection processing is performed in the image recognition unit 82B (see FIG. 12), and the papilla region information 95 is obtained. In addition, the image recognition unit 82B performs the image recognition processing using the trained model 84B (see FIG. 6) to obtain the intestinal tract direction information 90. The derivation unit 82C derives the papilla orientation information 102 based on the intestinal tract direction information 90. Then, the display image 94 is output to the display device 13 by the display control unit 82D. The display image 94 includes the papilla region N1 indicated by the papilla region information 95 and the papilla orientation ND indicated by the papilla orientation information 102. On the display device 13, the papilla region N1 and the papilla orientation ND are displayed on the screen 36. Accordingly, it is possible to make it easier for the user who observes the papilla N through the screen 36 to visually grasp the papilla orientation ND.

For example, in the ERCP examination, the camera 48 may be made to directly face the papilla N. In this case, by using the papilla orientation ND, it is easy to grasp the posture of the endoscope scope 18. In addition, in a case where a treatment tool is inserted into the papilla N, the papilla orientation ND is grasped, so that the treatment tool can be made to directly face the papilla N, and the treatment tool can be easily inserted into the papilla N.

### Second Embodiment

In the above first embodiment, the form example in which the intestinal tract direction CD is obtained by the image recognition processing on the intestinal wall image 41 has been described, but the technology of the present disclosure is not limited to this. In the present second embodiment, the intestinal wall image 41 is an image obtained by imaging the intestinal wall including the papilla N, and a rising direction RD of the papilla N is obtained by the image recognition processing on the intestinal wall image 41.

For example, in the ERCP examination, the camera 48 may made to directly face the papilla N in the rising direction RD. Accordingly, it is easy to estimate the running direction of the bile duct T and the pancreatic duct S extending from the papilla N, or it is easy to insert a treatment tool (for example, the cannula) into the papilla N. Thus, in the present second embodiment, the rising direction RD of the papilla N is acquired by the image recognition processing on the intestinal wall image 41.

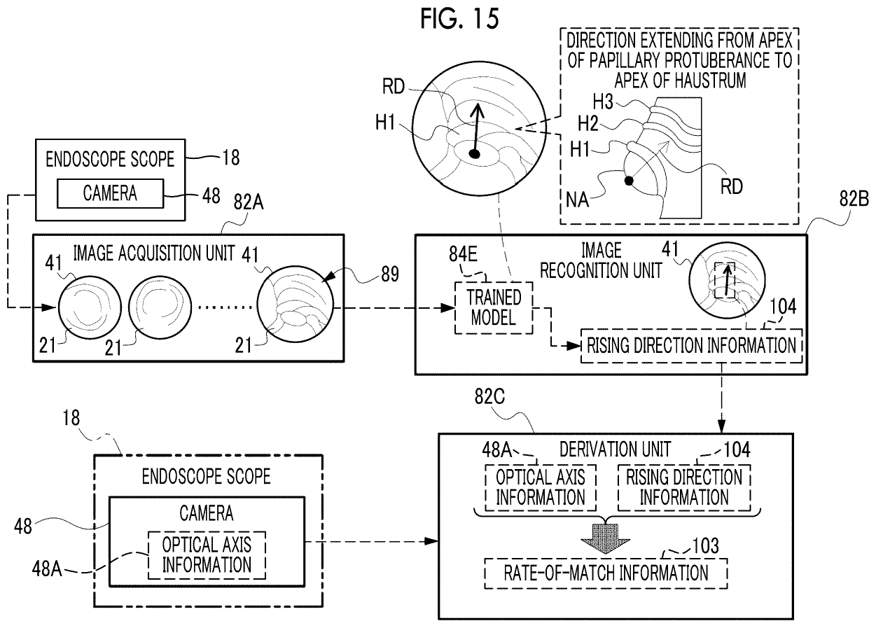

As shown in FIG. 15 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84E. Accordingly, the trained model 84E outputs rising direction information 104 corresponding to the input time-series image group 89. The image recognition unit 82B acquires the rising direction information 104 output from the trained model 84E. Here, the rising direction information 104 is information (for example, a position coordinate group of an axis line indicating the rising direction RD) capable of specifying the direction in which the papilla N protrudes.

The trained model 84E is obtained by performing machine learning using training data on the neural network to optimize the neural network. The training data is a plurality of pieces of data (that is, a plurality of frames of data) in which example data and correct answer data are associated with each other. The training data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the rising direction RD of the papilla N.

Here, the rising direction RD of the papilla N is specified, for example, as a direction extending from the apex of the papillary protuberance NA of the papilla N to the apex of a haustrum H1. This is because, according to the medical findings, the rising direction RD of the papilla N is often the same as the direction extending from the apex of the papillary protuberance NA to the apex of the haustrum H1. Here, in the papilla N, a plurality of folds (for example, folds H1 to H3) are present around a protruding part. The haustrum H1 is a fold closest to the papillary protuberance NA. Thus, as an example of the annotation in the correct answer data, an annotation in which a direction passing through the apex of the haustrum H1 is defined as the rising direction RD is used.

The derivation unit 82C derives the rate of match between the rising direction RD and the direction of the optical axis of the camera 48. The fact that the direction of the rising direction RD matches the direction of the optical axis means that the direction in which the camera 48 is directed directly faces the papilla N. That is, this means a state in which the distal end part 46 provided with the camera 48 is not in a direction (for example, a direction inclined with respect to the rising direction RD of the papilla N) that is not intended by the user.

Thus, the derivation unit 82C acquires the rising direction information 104 from the image recognition unit 82B. In addition, the derivation unit 82C acquires optical axis information 48A from the camera 48 of the endoscope scope 18. Then, the derivation unit 82C generates rate-of-match information 103 by comparing the direction indicated by the vertical direction information 97 with the direction of the optical axis indicated by the optical axis information 48A. The rate-of-match information 103 is information indicating the rate of match (for example, an angle formed by the direction of the optical axis and the rising direction RD) between the direction of the optical axis and the rising direction RD.

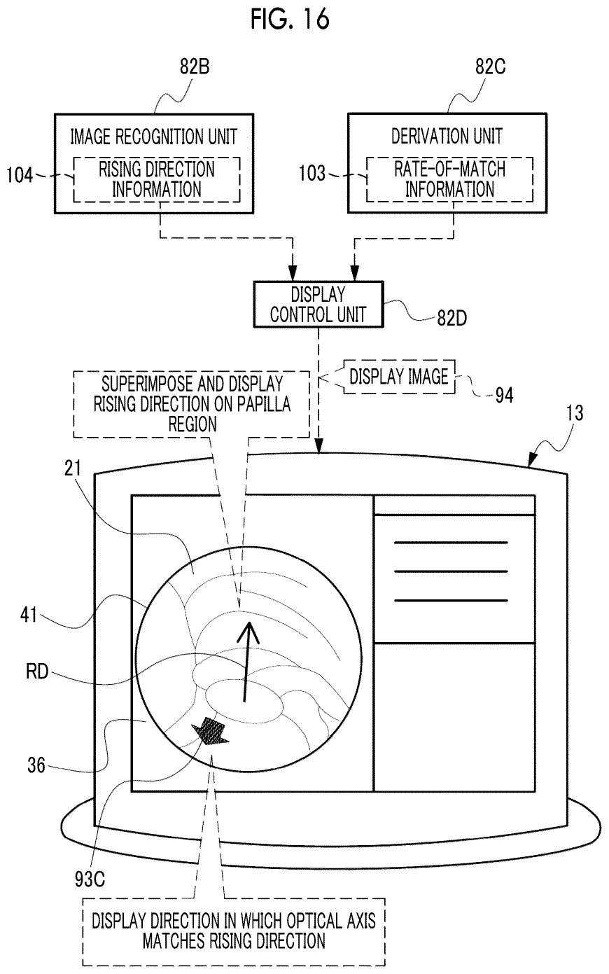

As shown in FIG. 16 as an example, the display control unit 82D acquires the rising direction information 104 from the image recognition unit 82B. In addition, the display control unit 82D acquires the rate-of-match information 103 from the derivation unit 82C. The display control unit 82D generates an operation instruction image 93C (for example, an arrow indicating an operation direction) for matching the direction of the optical axis with the rising direction RD according to the rate of match between the direction of the optical axis indicated by the rate-of-match information 103 and the rising direction RD. Then, the display control unit 82D generates a display image 94 including the rising direction RD indicated by the rising direction information 104, the operation instruction image 93C, and the intestinal wall image 41, and outputs the display image 94 to the display device 13. In the example shown in FIG. 16, the intestinal wall image 41 on which the rising direction RD and the operation instruction image 93C are superimposed and displayed on the screen 36 is shown on the display device 13.

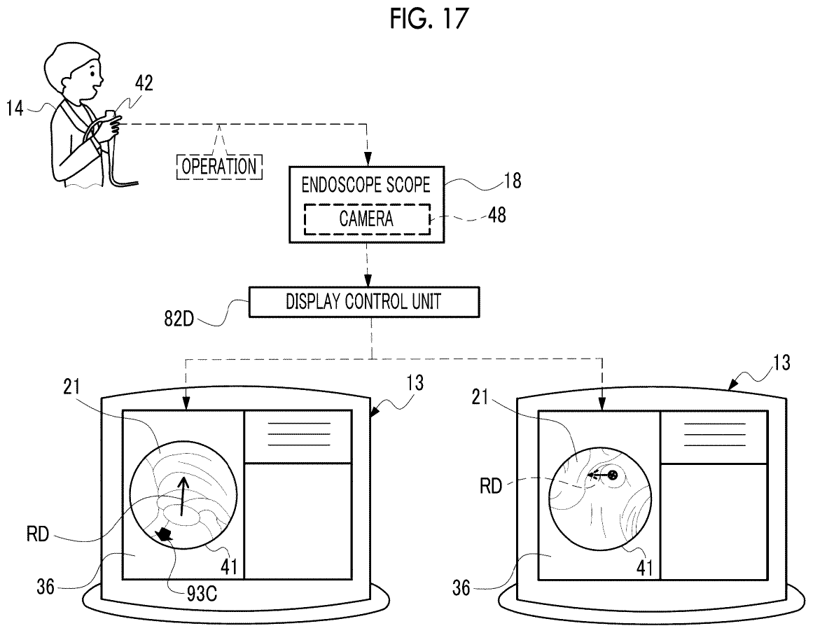

As shown in FIG. 17 as an example, the doctor 14 operates the endoscope scope 18 to bring the optical axis of the camera 48 close to the rising direction RD. Accordingly, since the intestinal wall image 41 in a case where the papilla N and the camera 48 directly face each other is obtained, it is easy to estimate the running direction of the bile duct T and the pancreatic duct S extending from the papilla N, or it is easy to insert a treatment tool (for example, the cannula) into the papilla N.

As described above, in the duodenoscope system 10 according to the present second embodiment, in the image recognition unit 82B of the processor 82, the image recognition processing is performed on the intestinal wall image 41, and as a result of the image recognition processing, the rising direction RD of the papilla N in the intestinal wall image 41 is detected. Then, the rising direction information 104 indicating the rising direction RD is output to the display control unit 82D, and the display image 94 generated by the display control unit 82D is output to the display device 13. The display image 94 includes the rising direction RD superimposed and displayed on the intestinal wall image 41. In this way, the display device 13 displays the rising direction RD on the screen 36. Accordingly, the user who observes the intestinal wall image 41 can visually grasp the rising direction RD of the papilla N.

In addition, in the duodenoscope system 10 according to the present second embodiment, the image recognition unit 82B obtains the rising direction information 104 based on the intestinal wall image 41. The rising direction information 104 is output to the display control unit 82D, and the display image 94 generated by the display control unit 82D is output to the display device 13. The display image 94 includes a display based on the rising direction information 104. Accordingly, the user who observes the intestinal wall image 41 can visually grasp the rising direction RD of the papilla N.

In addition, in the duodenoscope system 10 according to the present second embodiment, the display control unit 82D generates the display image 94. The display image 94 includes an image of an arrow indicating the rising direction RD. Accordingly, the user who observes the intestinal wall image 41 can be made to visually grasp the rising direction RD of the papilla N by visualizing the rising direction RD.

In addition, in the duodenoscope system 10 according to the present second embodiment, the derivation unit 82C acquires the optical axis information 48A from the camera 48. In addition, the derivation unit 82C generates the rate-of-match information 103 based on the optical axis information 48A and the rising direction information 104. The display control unit 82D generates the display image 94 based on the rate-of-match information 103 and outputs the display image 94 to the display device 13. The display image 94 includes a display related to the rate of match between the direction of the optical axis indicated by the rate-of-match information 103 and the rising direction RD. Accordingly, the user who observes the intestinal wall image 41 can be made to visually grasp the rate of match between the rising direction RD of the papilla N and the optical axis direction. For example, in a case where the optical axis matches the rising direction RD, there is a high probability that the camera 48 directly faces the papilla N. By holding the posture of the endoscope scope 18 in this state, the papilla N is easily observed, and a treatment tool is easily inserted into the papilla N.

In addition, in the duodenoscope system 10 according to the present second embodiment, in the image recognition processing of the image recognition unit 82B, the rising direction RD is specified as a direction extending from the apex of the papillary protuberance NA of the papilla N to the haustrum H1. Then, the display image 94 generated in the display control unit 82D is output to the display device 13. The display image 94 includes the rising direction RD. Accordingly, the user who observes the intestinal wall image 41 can visually grasp the direction extending from the opening of the papillary protuberance NA to the apex of the haustrum H1. As a result, it is possible to easily specify the running direction TD of the bile duct leading to the opening of the papilla N.

In addition, in the duodenoscope system 10 according to the present second embodiment, in the image recognition processing of the image recognition unit 82B, the rising direction RD is specified as a direction extending from the apex of the papillary protuberance NA of the papilla N to the haustrum H1. Then, the display image 94 generated in the display control unit 82D is output to the display device 13. The display image 94 includes an image of an arrow indicating the rising direction RD. Accordingly, the user who observes the intestinal wall image 41 can visually grasp the direction extending from the opening of the papillary protuberance NA to the apex of the haustrum H1. As a result, it is possible to easily specify the running direction TD of the bile duct leading to the opening of the papilla N.

In addition, in the duodenoscope system 10 according to the present second embodiment, the image recognition unit 82B performs the image recognition processing on the intestinal wall image 41 to obtain the rising direction information 104 indicating the rising direction RD. Accordingly, the rising direction information 104 with higher accuracy is obtained compared to a case where the user designates the rising direction RD with respect to the intestinal wall image 41 by visual observation.

### Fifth Modification Example

In the above second embodiment, the form example in which the rising direction RD is specified as a direction extending from the apex of the papillary protuberance NA to the apex of the haustrum H1 has been described, but the technology of the present disclosure is not limited to this. The rising direction RD is specified based on the aspects of the plurality of folds H1 to H3.

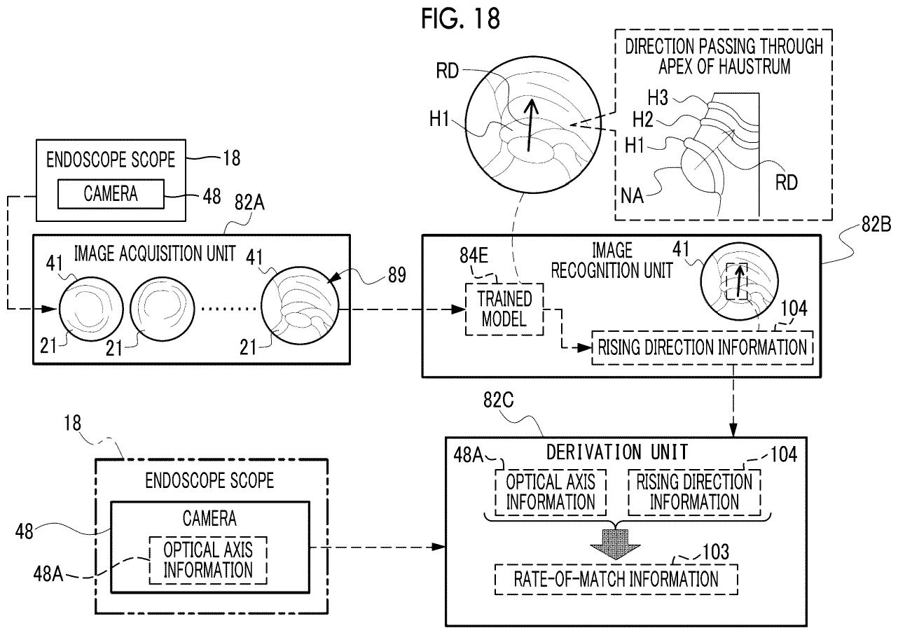

As shown in FIG. 18 as an example, the image recognition unit 82B acquires the time-series image group 89 from the image acquisition unit 82A and inputs the acquired time-series image group 89 to the trained model 84E. Accordingly, the trained model 84E outputs rising direction information 104 corresponding to the input time-series image group 89. The image recognition unit 82B acquires the rising direction information 104 output from the trained model 84E.

Here, the rising direction RD of the papilla N is specified, for example, as the direction passing through the apex of the haustrum H1. According to the medical findings, the rising direction RD of the papilla N may match the direction passing through the apex of the haustrum H1. Thus, as an example of the annotation in the correct answer data, an annotation in which a direction passing through the apex of the haustrum H1 is defined as the rising direction RD is used.

Here, a form example in which the rising direction RD is specified as the direction passing through the apex of the haustrum H1 has been described, but this is merely an example. The rising direction RD may be specified as a direction passing through at least one of the apexes of the plurality of folds H1 to H3.

As described above, in the duodenoscope system 10 according to the present fifth modification example, in the image recognition processing in the image recognition unit 82B, the rising direction RD is specified based on the aspects of the plurality of folds H1 to H3. Then, the display image 94 generated in the display control unit 82D is output to the display device 13. The display image 94 includes the rising direction RD. Accordingly, the user who observes the intestinal wall image 41 can visually grasp the direction passing through the apex of the haustrum H1 of the papillary protuberance NA as the rising direction RD.

### Sixth Modification Example

In the above second embodiment, the form example in which the rising direction RD is specified as a direction extending from the apex of the papillary protuberance NA to the apex of the haustrum H1 has been described, but the technology of the present disclosure is not limited to this. In the present sixth modification example, the rising direction RD is specified based on the papillary protuberance NA and the plurality of folds H1 to H3.

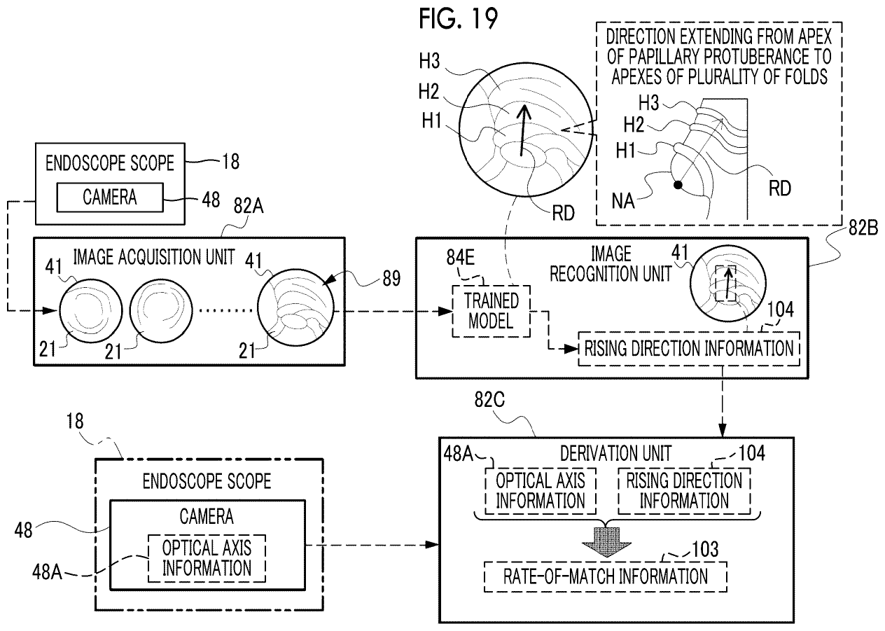

As shown in FIG. 19 as an example, the image recognition unit 82B acquires the time-series image group 89 from the image acquisition unit 82A and inputs the acquired time-series image group 89 to the trained model 84E. Accordingly, the trained model 84E outputs rising direction information 104 corresponding to the input time-series image group 89. The image recognition unit 82B acquires the rising direction information 104 output from the trained model 84E.

Here, the rising direction RD of the papilla N is specified, for example, as the direction passing through the respective apexes of the haustrum H1, the folds H2, and H3 from the apex of the papillary protuberance NA. According to the medical findings, the rising direction RD of the papilla N may match the direction passing through the apexes of the haustrum H1, the folds H2, and H3 from the apex of the papillary protuberance NA. Thus, as an example of the annotation in the correct answer data, an annotation in which the direction passing through the apexes of the haustrum H1, the folds H2, and H3 from the apex of the papillary protuberance NA is defined as the rising direction RD is used.

As described above, in the duodenoscope system 10 according to the present sixth modification example, in the image recognition processing of the image recognition unit 82B, the rising direction RD is specified based on the papillary protuberance NA and the plurality of folds H1 to H3. Then, the display image 94 generated in the display control unit 82D is output to the display device 13. The display image 94 includes the rising direction RD. Accordingly, the user who observes the intestinal wall image 41 can visually grasp the direction passing through the apex of the papillary protuberance NA and the apexes of the plurality of folds H1 to H3 as the rising direction RD.

### Seventh Modification Example

In the above second embodiment, the form example in which the rising direction RD is obtained by the image recognition processing on the intestinal wall image 41 has been described, but the technology of the present disclosure is not limited to this. In the present seventh modification example, the running direction TD of the bile duct is obtained based on the rising direction RD.

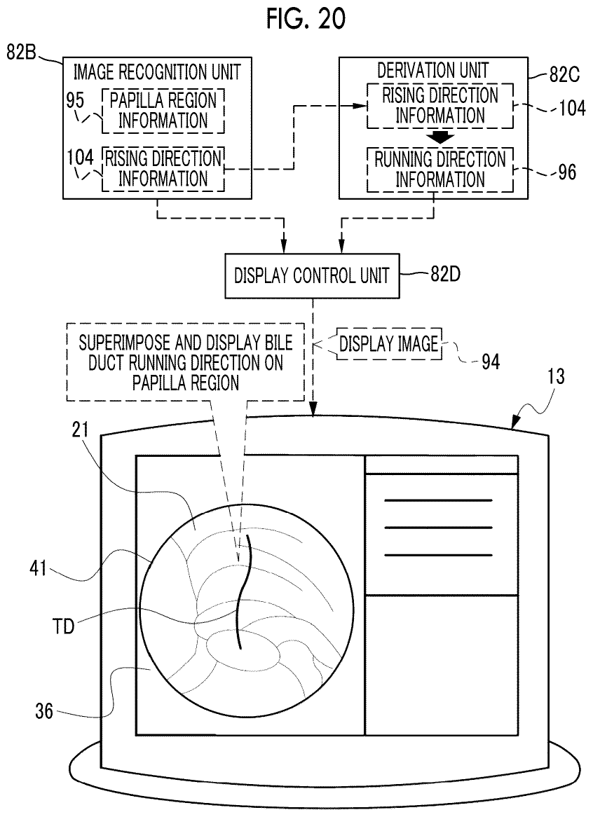

The image recognition unit 82B performs the image recognition processing on the intestinal wall image 41 to obtain the rising direction information 104 and the papilla region information 95 (see FIGS. 12 and 15). As shown in FIG. 20 as an example, the derivation unit 82C derives the running direction information 96 based on the rising direction information 104. The running direction TD of the bile duct has a predetermined orientation relationship with the rising direction RD of the papilla N. Specifically, the derivation unit 82C derives the running direction TD as a direction of 11 o'clock in a case where the rising direction RD is a direction of 12 o'clock.

The display control unit 82D acquires the running direction information 96 from the derivation unit 82C. The display control unit 82D generates the display image 94 on which the running direction TD indicated by the running direction information 96 is superimposed and displayed on the intestinal wall image 41 acquired from the image acquisition unit 82A (see FIG. 6), and outputs the display image 94 to the display device 13. On the display device 13, the intestinal wall image 41 on which the running direction TD is superimposed and displayed is displayed on the screen 36.

As described above, in the duodenoscope system 10 according to the present seventh modification example, the running direction information 96 is obtained based on the rising direction information 104 in the derivation unit 82C. In this way, since the running direction information 96 is obtained by the rising direction information 104, it is easy to specify the running direction TD compared to a case where the running direction information 96 is obtained by the image recognition processing.

In addition, in the duodenoscope system 10 according to the present seventh modification example, the display control unit 82D generates the display image 94. The display image 94 includes an image showing the running direction TD. Accordingly, the user who observes the intestinal wall image 41 can visually grasp the running direction TD of the bile duct.

### Eighth Modification Example

In the above second embodiment, the form example in which the rising direction RD of the papilla N is obtained by the image recognition processing on the intestinal wall image 41 has been described, but the technology of the present disclosure is not limited to this. In the present eighth modification example, a direction MD (hereinafter, also simply referred to as a “plane direction MD”) of a plane in which an opening is present at the papilla N is obtained by the image recognition processing on the intestinal wall image 41.

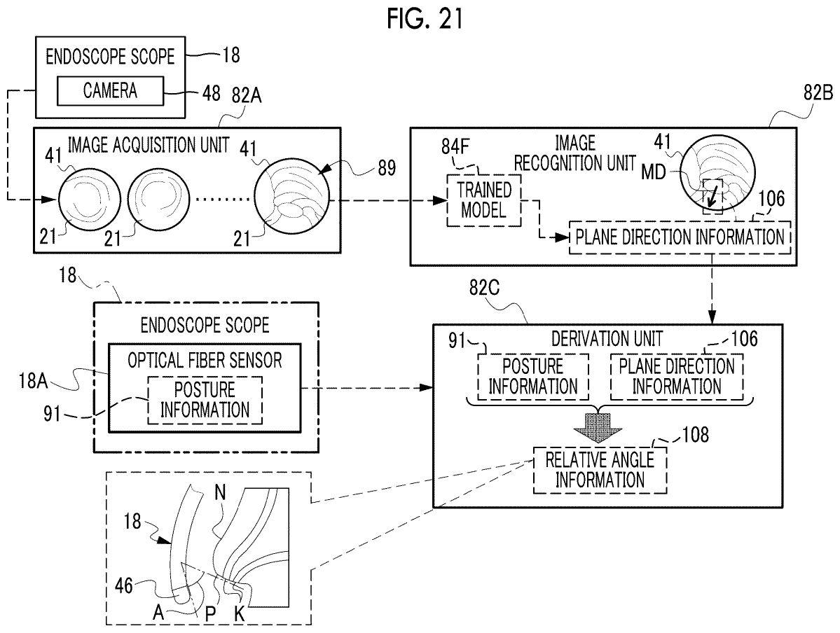

As shown in FIG. 21 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84F. Accordingly, the trained model 84F outputs plane direction information 106 corresponding to the input time-series image group 89. The image recognition unit 82B acquires the plane direction information 106 output from the trained model 84F. Here, the plane direction information 106 is information (for example, a position coordinate group of an axis line indicating the plane direction MD) capable of specifying the plane direction MD.

The trained model 84F is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the plane direction MD.

The derivation unit 82C derives a relative angle between a plane P on which an opening K of the papilla N is provided and the posture of the endoscope scope 18. The fact that the relative angle between the plane P provided with the opening K of the papilla N and the posture of the endoscope scope 18 approaches 0 means that the camera 48 approaches a state in which the camera 48 directly faces the papilla N. Thus, the derivation unit 82C acquires the plane direction information 106 from the image recognition unit 82B. In addition, the derivation unit 82C acquires the posture information 91 from the optical fiber sensor 18A of the endoscope scope 18. Then, the derivation unit 82C generates relative angle information 108 by comparing the plane P having the opening K from the orientation of the plane indicated by the plane direction information 106 with the posture of the endoscope scope 18 indicated by the posture information 91. The relative angle information 108 is information indicating an angle A formed by the plane P and the posture (for example, the imaging surface of the camera 48) of the endoscope scope 18.

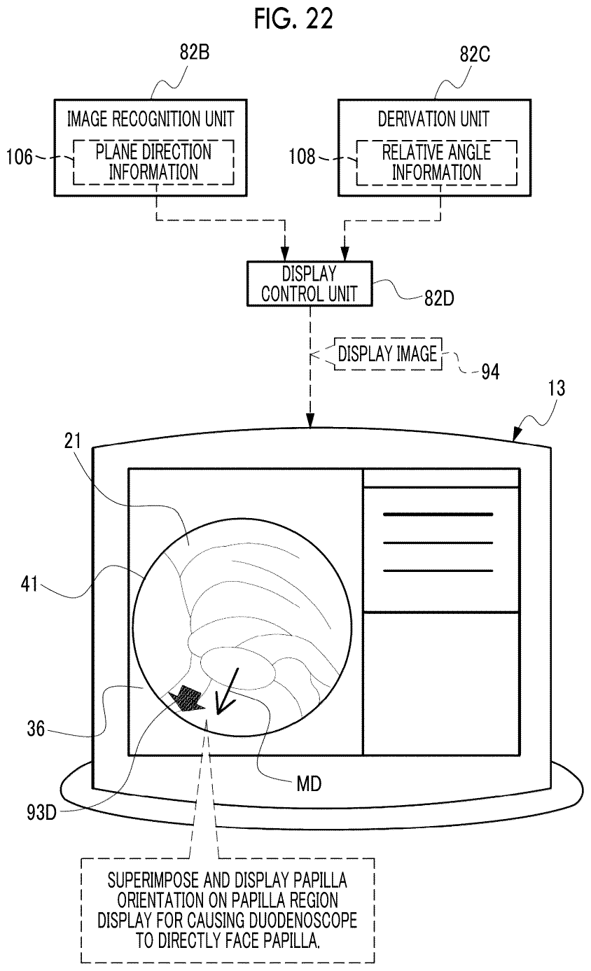

As shown in FIG. 22 as an example, the display control unit 82D acquires the plane direction information 106 from the image recognition unit 82B. In addition, the display control unit 82D acquires the relative angle information 108 from the derivation unit 82C. The display control unit 82D generates an operation instruction image 93D (for example, an arrow indicating an operation direction) for causing the camera 48 to directly face the papilla N according to the angle indicated by the relative angle information 108. Then, the display control unit 82D generates a display image 94 including the plane direction MD indicated by the plane direction information 106, the operation instruction image 93D, and the intestinal wall image 41, and outputs the display image 94 to the display device 13. In the example shown in FIG. 22, the intestinal wall image 41 on which the plane direction MD and the operation instruction image 93D are superimposed and displayed on the screen 36 is shown on the display device 13.

As described above, in the duodenoscope system 10 according to the present eighth modification example, the image recognition unit 82B of the processor 82 performs the image recognition processing on the intestinal wall image 41 and detects the plane direction MD of the papilla N in the intestinal wall image 41 as a result of the image recognition processing. Then, the plane direction information 106 indicating the plane direction MD is output to the display control unit 82D, and the display image 94 generated in the display control unit 82D is output to the display device 13. The display image 94 includes the plane direction MD superimposed and displayed on the intestinal wall image 41. In this way, the plane direction MD is displayed on the screen 36 of the display device 13. Accordingly, the user who observes the intestinal wall image 41 can visually grasp the plane direction MD of the papilla N.

In addition, in the duodenoscope system 10 according to the present eighth modification example, the derivation unit 82C acquires the posture information 91, which is information capable of specifying the posture of the endoscope scope 18, from the optical fiber sensor 18A. In addition, the derivation unit 82C generates the relative angle information 108 based on the posture information 91 and the plane direction information 106. Moreover, in the display control unit 82D, the operation instruction image 93D for causing the camera 48 to directly face the papilla N is generated based on the relative angle information 108. The display control unit 82D outputs the operation instruction image 93D to the display device 13, and the operation instruction image 93D is superimposed and displayed on the intestinal wall image 41 on the display device 13. Accordingly, in a state in which the endoscope scope 18 is inserted into the duodenum, it is easy for the user to set the posture of the endoscope scope 18 with respect to the plane direction MD of the papilla N to an intended posture.

### Ninth Modification Example

In the above second embodiment, the form example in which the rising direction RD obtained by the image recognition processing on the intestinal wall image 41 is displayed has been described, but the technology of the present disclosure is not limited to this. In the present ninth modification example, a papilla plane image 93E is displayed.

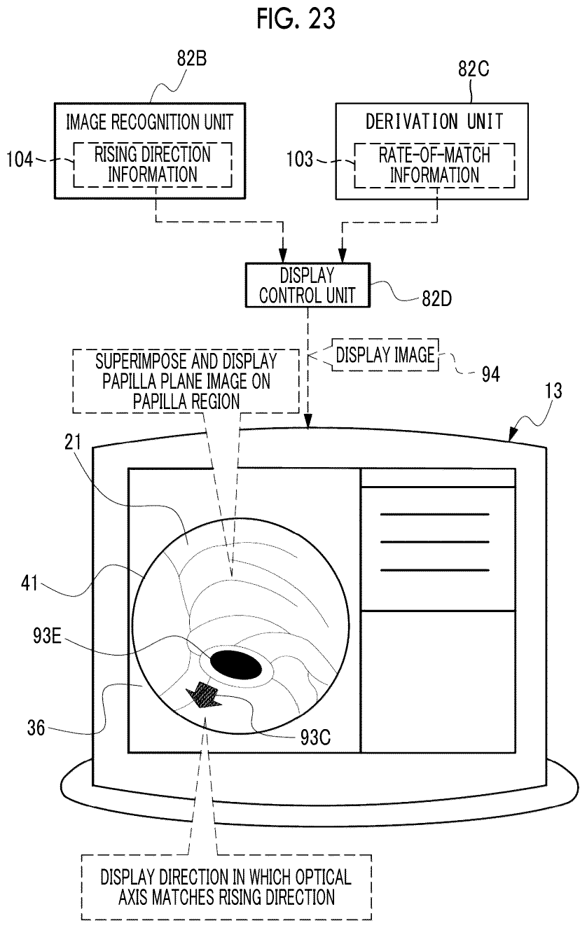

As shown in FIG. 23 as an example, the display control unit 82D acquires the rising direction information 104 from the image recognition unit 82B. The display control unit 82D generates the papilla plane image 93E based on the rising direction RD indicated by the rising direction information 104. The papilla plane image 93E is an image capable of specifying a plane intersecting the rising direction RD at a predetermined angle (for example, 90 degrees). Moreover, the display control unit 82D adjusts the papilla plane image 93E to a size and a shape corresponding to the papilla region N1 based on the papilla region information 95 obtained in the image recognition unit 82B. In addition, the display control unit 82D generates the operation instruction image 93C.

Then, the display control unit 82D generates a display image 94 including the papilla plane image 93E, the operation instruction image 93C, and the intestinal wall image 41, and outputs the display image 94 to the display device 13. In the example shown in FIG. 23, the intestinal wall image 41 on which the papilla plane image 93E and the operation instruction image 93C are superimposed and displayed on the screen 36 is shown on the display device 13.

As described above, in the duodenoscope system 10 according to the present ninth modification example, the papilla plane image 93E is generated in the display control unit 82D based on the rising direction information 104. The display control unit 82D outputs the papilla plane image 93E to the display device 13, and the papilla plane image 93E is superimposed and displayed on the intestinal wall image 41 on the display device 13. Accordingly, it is easy for the user who observes the intestinal wall image 41 to visually predict the position of an opening included in the papilla N.

### Third Embodiment

The form example in which, in the above first embodiment, the intestinal tract direction CD is obtained by the image recognition processing on the intestinal wall image 41, and in the above second embodiment, the rising direction RD is obtained by the image recognition processing on the intestinal wall image 41 has been described, but the technology of the present disclosure is not limited to this. In the present third embodiment, the running direction TD of the bile duct T is obtained by the image recognition processing on the intestinal wall image 41.

For example, in the ERCP examination, a treatment tool (for example, the cannula) may be inserted into the papilla N, and the treatment tool may be intubated into the bile duct T or the pancreatic duct S in the papilla N. In this case, in the intestinal wall image 41, it is difficult to grasp the running direction of the bile duct T or the pancreatic duct S present inside the papilla N. Thus, in the present third embodiment, the running direction of the bile duct T or the pancreatic duct S is acquired by the image recognition processing on the intestinal wall image 41. In addition, in the following, for convenience of description, the case of the bile duct T will be described as an example.

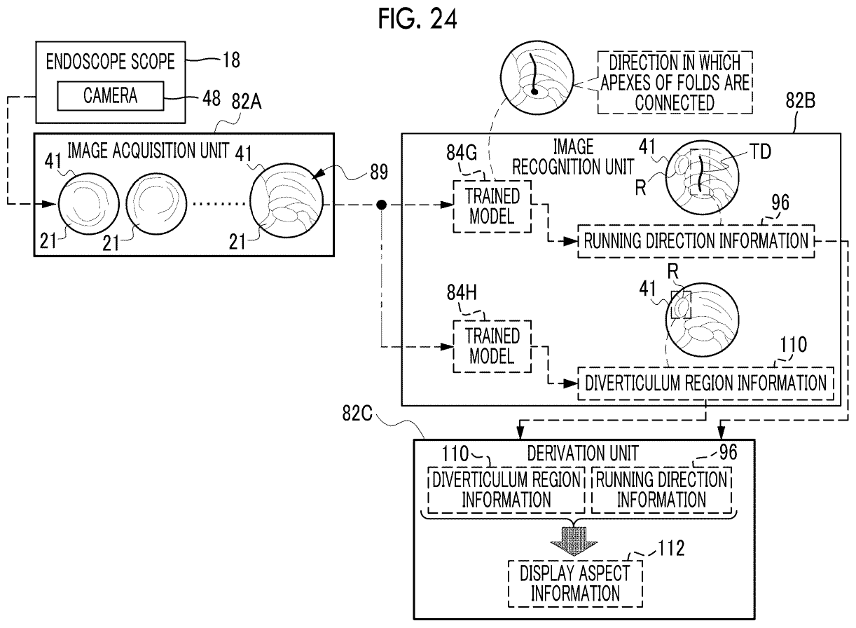

As shown in FIG. 24 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84G. Accordingly, the trained model 84G outputs the running direction information 96 corresponding to the input time-series image group 89. The image recognition unit 82B acquires the running direction information 96 output from the trained model 84G.

The trained model 84G is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the running direction TD.

Here, the running direction TD of the bile duct T is specified as, for example, the direction passing through the apexes of the plurality of folds of the papilla N. This is because, according to the medical findings, the running direction of the bile duct T may match a line connecting the apex of a fold. Thus, as an example of the annotation in the correct answer data, an annotation in which a direction passing through an apex of a fold of the papilla N is set as the running direction TD of the bile duct T is used.

In addition, the acquired time-series image group 89 is input to a trained model 84H. Accordingly, the trained model 84H outputs diverticulum region information 110 corresponding to the input time-series image group 89. The image recognition unit 82B acquires the diverticulum region information 110 output from the trained model 84H. The diverticulum region information 110 is information (coordinates indicating the size and position of the diverticulum) capable of specifying a region indicating a diverticulum present in the papilla N. Here, the diverticulum is a region in which a part of the papilla N protrudes in a pouch-like shape to the outside of the duodenum.

The trained model 84H is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying a region indicating a diverticulum.

The derivation unit 82C derives an aspect for displaying the running direction TD. For example, the running direction TD is specified in an aspect in which the diverticulum is avoided. This is because, according to the medical findings, the running direction TD may be formed to avoid the diverticulum. Thus, the derivation unit 82C changes the display aspect of the running direction TD based on the diverticulum region information 110. Specifically, the derivation unit 82C changes a portion intersecting the diverticulum, which is indicated by the diverticulum region information 110, in the running direction TD indicated by the running direction information 96 to an aspect in which the diverticulum is avoided. In this way, the derivation unit 82C generates display aspect information 112 indicating the display aspect of the changed running direction TD.

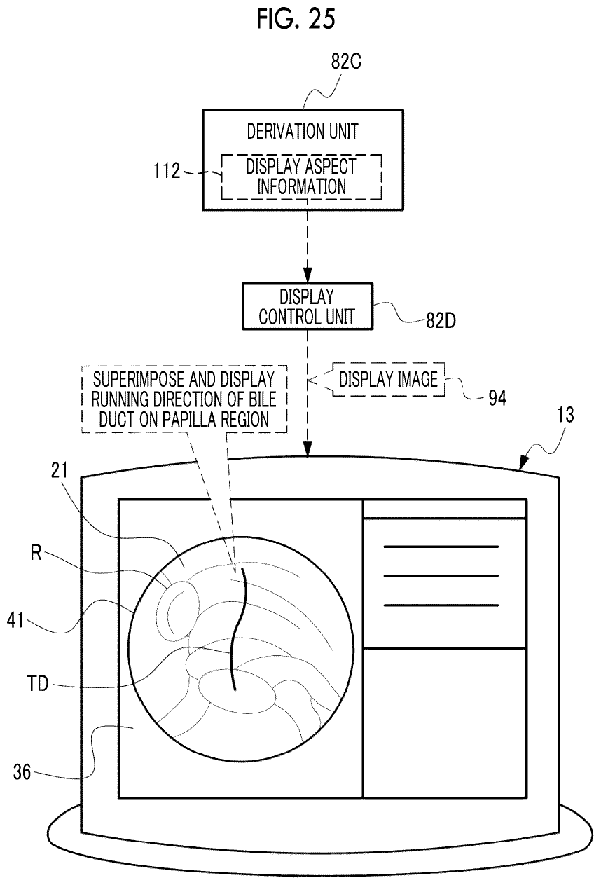

As shown in FIG. 25 as an example, the display control unit 82D acquires the display aspect information 112 from the derivation unit 82C. The display control unit 82D generates a display image 94 including the changed running direction TD and the intestinal wall image 41 indicated by the display aspect information 112, and outputs the display image 94 to the display device 13. In the example shown in FIG. 25, the intestinal wall image 41 on which the changed running direction TD is superimposed and displayed on the screen 36 is shown on the display device 13.

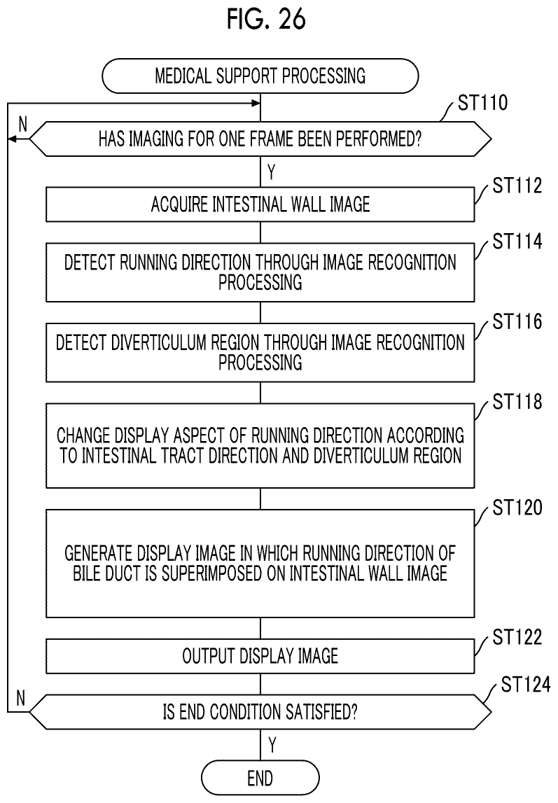

Next, the operation of a portion of the duodenoscope system 10 according to the technology of the present disclosure will be described with reference to FIG. 26.

FIG. 26 shows an example of a flow of the medical support processing performed by the processor 82.

In the medical support processing shown in FIG. 26, first, in step ST110, the image acquisition unit 82A determines whether or not imaging for one frame has been performed by the camera 48 provided in the endoscope scope 18. In a case where the imaging for one frame has not been performed by the camera 48 in step ST110, the determination result is “No”, and the determination in step ST110 is performed again. In a case where the imaging for one frame has been performed by the camera 48 in step ST110, the determination result is “Yes”, and the medical support processing proceeds to step ST112.

In step ST112, the image acquisition unit 82A acquires one frame of the intestinal wall image 41 from the camera 48 provided in the endoscope scope 18. After the processing in step ST112 is executed, the medical support processing proceeds to step ST114.

In step ST114, the image recognition unit 82B performs image recognition processing (that is, image recognition processing using the trained model 84G) using the AI method on the intestinal wall image 41 acquired in step ST112 to detect the running direction TD. After the processing in step ST114 is executed, the medical support processing proceeds to step ST116.

In step ST116, the image recognition unit 82B detects the diverticulum region by performing the image recognition processing (that is, the image recognition processing using the trained model 84H) using the AI method on the intestinal wall image 41 acquired in step ST112. After the processing in step ST116 is executed, the medical support processing proceeds to step ST118.

In step ST118, the derivation unit 82C changes the display aspect of the running direction TD based on the running direction TD obtained by the image recognition unit 82B in step ST114 and the diverticulum region obtained by the image recognition unit 82B in step ST116. Specifically, the derivation unit 82C changes the display aspect of the running direction TD to an aspect in which the diverticulum region is avoided. After the processing in step ST118 is executed, the medical support processing proceeds to step ST120.

In step ST120, the display control unit 82D generates the display image 94 on which the running direction TD of which the display aspect is changed by the derivation unit 82C in step ST118 is superimposed and displayed on the intestinal wall image 41. After the processing in step ST120 is executed, the medical support processing proceeds to step ST122.

In step ST122, the display control unit 82D outputs the display image 94 generated in step ST120 to the display device 13. After the processing in step ST122 is executed, the medical support processing proceeds to step ST124.

In step ST124, the display control unit 82D determines whether or not a condition for ending the medical support processing is satisfied. An example of the medical support processing end condition is a condition (for example, a condition in which an instruction to end the medical support processing is received by the receiving device 62) in which an instruction to end the medical support processing is issued to the duodenoscope system 10.

In a case where the medical support processing end condition is not satisfied in step ST124, the determination result is “No”, and the medical support processing proceeds to step ST110. In a case where the medical support processing end condition is satisfied in step ST124, the determination result is “Yes”, and the medical support processing ends.

As described above, in the duodenoscope system 10 according to the present third embodiment, the image recognition processing is performed on the intestinal wall image 41 in the image recognition unit 82B of the processor 82, and the running direction TD of the bile duct in the intestinal wall image 41 is detected as a result of the image recognition processing. Then, the running direction information 96 indicating the running direction TD is output to the display control unit 82D, and the display image 94 generated in the display control unit 82D is output to the display device 13. The display image 94 includes the running direction TD superimposed and displayed on the intestinal wall image 41. In this way, the running direction TD is displayed on the screen 36 of the display device 13. Accordingly, the user who observes the intestinal wall image 41 can visually grasp the running direction TD of the bile duct.

In addition, in the duodenoscope system 10 according to the present third embodiment, the image recognition unit 82B performs the image recognition processing on the intestinal wall image 41 to obtain the diverticulum region information 110. The derivation unit 82C generates the display aspect information 112 based on the running direction information 96 and the diverticulum region information 110. Then, the display aspect information 112 indicating the changed running direction TD is output to the display control unit 82D, and the display image 94 generated in the display control unit 82D is output to the display device 13. The display image 94 includes the changed running direction TD superimposed and displayed on the intestinal wall image 41. In this way, the changed running direction TD is displayed on the screen 36 of the display device 13. Accordingly, the user who observes the intestinal wall image 41 can visually grasp the running direction TD of the bile duct changed according to the presence of the diverticulum. For example, it is possible to suppress the occurrence of a situation in which the user who observes the intestinal wall image 41 is made to visually erroneously grasp the running direction TD of the bile duct leading to the opening of the papilla N due to the presence of the diverticulum.

In addition, in the duodenoscope system 10 according to the present third embodiment, in the derivation unit 82C, the display aspect information 112 indicates the changed running direction TD in an aspect in which the diverticulum is avoided in the running direction TD indicated by the running direction information 96. Then, the changed running direction TD is displayed on the screen 36 of the display device 13. Accordingly, the user who observes the intestinal wall image 41 can visually grasp the running direction TD of the bile duct changed in the aspect in which the diverticulum is avoided.

In addition, in the above third embodiment, the aspect in which the diverticulum is avoided is exemplified as the form example in which the display aspect of the running direction TD of the bile duct is changed, but the technology of the present disclosure is not limited to this. For example, in the running direction TD of the bile duct, a region intersecting the diverticulum may be hidden, or a region intersecting the diverticulum may be represented by a broken line or may be semi-translucent.

In addition, in the above third embodiment, the form example in which the diverticulum is detected by the image recognition processing on the intestinal wall image 41 and the display aspect of the running direction TD is changed according to the diverticulum has been described, but the technology of the present disclosure is not limited to this. For example, an aspect in which the detection of the diverticulum is not performed may be adopted.

### Tenth Modification Example

In the above third embodiment, the form example in which the running direction TD of the bile duct is displayed by avoiding the diverticulum has been described, but the technology of the present disclosure is not limited to this. In the present tenth modification example, in a case where the running direction TD of the bile duct intersects the diverticulum, the user is notified of the fact.

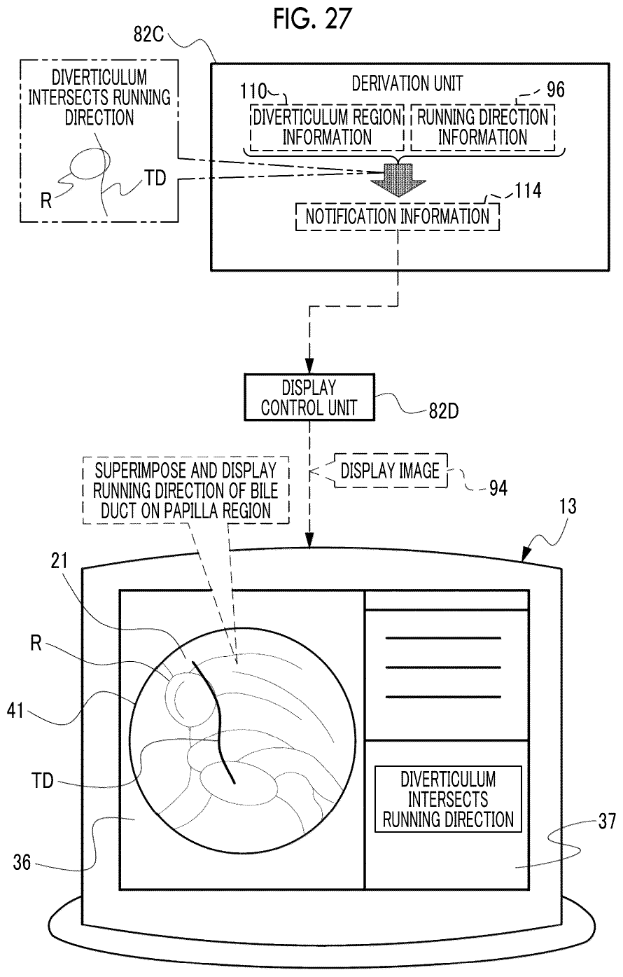

As shown in FIG. 27 as an example, the derivation unit 82C acquires the running direction information 96 and the diverticulum region information 110 from the image recognition unit 82B. The derivation unit 82C specifies a positional relationship between the diverticulum and the running direction TD based on the diverticulum region information 110 and the running direction information 96. Specifically, the derivation unit 82C specifies whether or not the diverticulum and the running direction TD intersect each other by comparing the running direction TD indicated by the running direction information 96 with the position and the size of the diverticulum indicated by the diverticulum region information 110. Then, in a case where the derivation unit 82C specifies that the running direction TD and the diverticulum have a positional relationship in which the running direction TD intersects the diverticulum, the derivation unit 82C generates notification information 114.

The derivation unit 82C outputs the notification information 114 to the display control unit 82D. In this case, the display control unit 82D generates the display image 94 including the content of notifying the user that the diverticulum indicated by the notification information 114 intersects the running direction TD. In the example shown in FIG. 27, on the display device 13, an example in which a message “the diverticulum intersects the running direction” is displayed on the screen 37 is shown.

As described above, in the duodenoscope system 10 according to the present tenth modification example, the derivation unit 82C specifies the positional relationship between the diverticulum and the running direction TD based on the diverticulum region information 110 and the running direction information 96, and generates the notification information 114 based on the identification result. In the display control unit 82D, the display image 94 is generated based on the notification information 114 and is output to the display device 13. The display image 94 includes a display indicating that the diverticulum indicated by the notification information 114 intersects the running direction. Accordingly, the user can be made to perceive that the diverticulum intersects the running direction. For example, it is possible to suppress the occurrence of a situation in which the user who observes the intestinal wall image 41 is made to visually erroneously grasp the running direction TD of the bile duct leading to the opening of the papilla N due to the presence of the diverticulum.

### Fourth Embodiment

In the above first embodiment to third embodiment, the form example in which the information related to biological tissue, such as the intestinal tract direction CD, the papilla N, and the running direction TD of the bile duct, is specified by the image recognition processing on the intestinal wall image 41 has been described, but the technology of the present disclosure is not limited to this. In the present fourth embodiment, a relationship between a treatment tool and the biological tissue is specified by performing the image recognition processing on the intestinal wall image 41.

For example, in the ERCP examination, various treatments (for example, the cannula is inserted into the papilla N) using a treatment tool may be performed on the papilla N. In this case, the positional relationship between the papilla N and the treatment tool affects the success or failure of the procedure. For example, in a case where the traveling direction of the treatment tool does not match the papilla orientation ND, the treatment tool cannot appropriately approach the papilla N, and it is difficult to succeed in the procedure. Thus, in the present fourth embodiment, the positional relationship between the treatment tool and the papilla N is specified by the image recognition processing on the intestinal wall image 41.

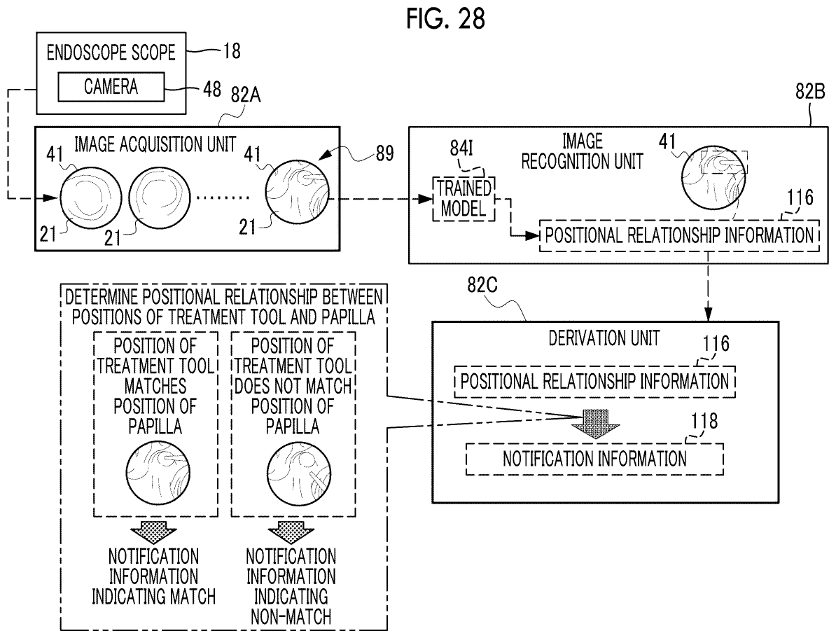

As shown in FIG. 28 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84I. Accordingly, the trained model 84I outputs positional relationship information 116 corresponding to the input time-series image group 89. The image recognition unit 82B acquires the positional relationship information 116 output from the trained model 84I. Here, the positional relationship information 116 is information (for example, a distance and an angle between the position of the papilla N and the position of the tip of the treatment tool) capable of specifying the position of the papilla N and the position of the treatment tool.

The trained model 84I is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation capable of specifying the position of the papilla N and the position of the treatment tool.

The derivation unit 82C acquires the positional relationship information 116 from the image recognition unit 82B. The derivation unit 82C generates notification information 118 that is information for notifying the user of the positional relationship between the papilla N and the treatment tool, based on the positional relationship information 116. The derivation unit 82C compares the position of the treatment tool indicated by the positional relationship information 116 with the position of the papilla N. Then, in a case where the position of the treatment tool matches the position of the papilla N, the derivation unit 82C generates the notification information 118 indicating that the position of the treatment tool matches the position of the papilla N. In addition, in a case where the position of the treatment tool does not match the position of the papilla N, the derivation unit 82C generates the notification information 118 indicating that the position of the treatment tool does not match the position of the papilla N.

Here, a case whether the position of the treatment tool matches the position of the papilla N has been described as an example, but this is merely an example. For example, whether or not the position of the treatment tool and the position of the papilla N are within a predetermined range (for example, within a predetermined distance and angle range) may be determined.

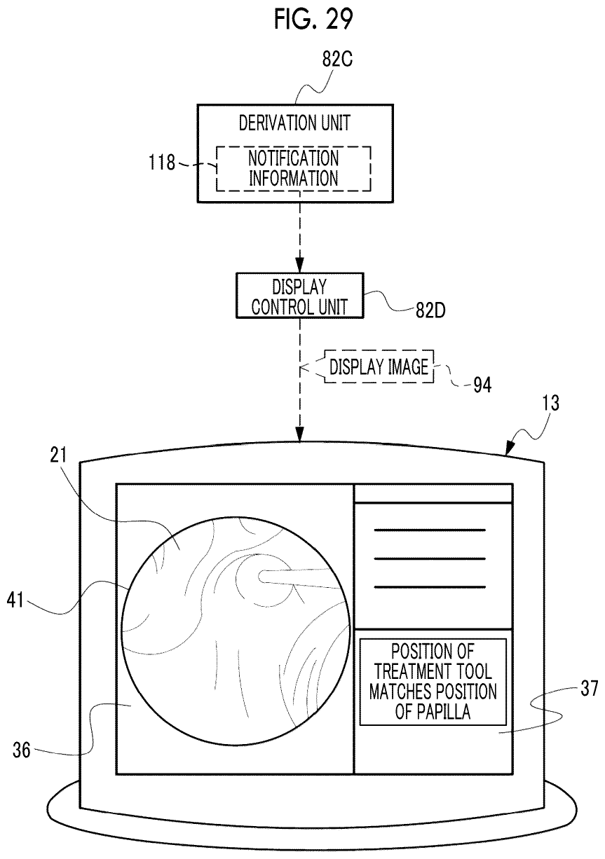

As shown in FIG. 29 as an example, the display control unit 82D acquires the notification information 118 from the derivation unit 82C. The derivation unit 82C outputs the notification information 118 to the display control unit 82D. In this case, the display control unit 82D generates the display image 94 including the content of notifying the user of the positional relationship between the treatment tool and the papilla N indicated by the notification information 118. In the example shown in FIG. 29, on the display device 13, an example in which a message “the position of the treatment tool and the position of the papilla match” is displayed on the screen 37 is shown.

As described above, in the duodenoscope system 10 according to the present fourth embodiment, the image recognition unit 82B of the processor 82 performs the image recognition processing on the intestinal wall image 41 and specifies the positional relationship between the treatment tool and the papilla. The derivation unit 82C performs the determination related to the positional relationship between the treatment tool and the papilla N based on the positional relationship information 116 indicating the positional relationship between the treatment tool and the papilla and generates the notification information 118 generated based on the determination result. In the display control unit 82D, the display image 94 is generated based on the notification information 118 and is output to the display device 13. The display image 94 includes a display related to the positional relationship between the treatment tool indicated by the notification information 118 and the papilla N. Accordingly, the user who observes the intestinal wall image 41 can be made to perceive the relationship between the position of the treatment tool and the position of the papilla N.

### Eleventh Modification Example

In the above fourth embodiment, the form example in which the relationship between the position of the papilla N and the position of the treatment tool is specified as the positional relationship between the treatment tool and the papilla N has been described, but the technology of the present disclosure is not limited to this. In the present eleventh modification example, the relationship between the traveling direction of the treatment tool and the papilla orientation ND is specified.

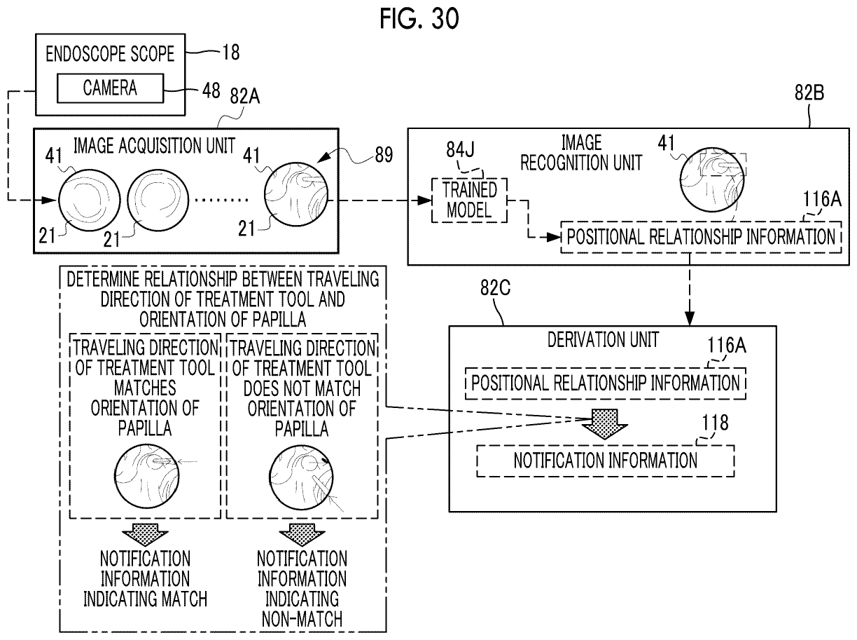

As shown in FIG. 30 as an example, the image recognition unit 82B acquires the time-series image group 89 from the image acquisition unit 82A and inputs the acquired time-series image group 89 to a trained model 84J. Accordingly, the trained model 84J outputs positional relationship information 116A corresponding to the input time-series image group 89. Here, the positional relationship information 116A is information (for example, an angle formed by the papilla orientation ND and the traveling direction of the treatment tool) capable of specifying the papilla orientation ND and the traveling direction of the treatment tool.

The trained model 84J is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the relationship between the papilla orientation ND and the traveling direction of the treatment tool.

The derivation unit 82C acquires the positional relationship information 116A from the image recognition unit 82B. The derivation unit 82C generates notification information 118 that is information for notifying the user of the positional relationship between the papilla N and the treatment tool, based on the positional relationship information 116A. In a case where the angle between the papilla orientation direction ND and the traveling direction of the treatment tool is within a predetermined range, the derivation unit 82C generates the notification information 118 indicating that the papilla orientation direction ND matches the traveling direction of the treatment tool. In addition, in a case where the angle between the papilla orientation direction ND and the traveling direction of the treatment tool exceeds a predetermined range, the derivation unit 82C generates the notification information 118 indicating that the papilla orientation direction ND does not match the traveling direction of the treatment tool.

As described above, in the duodenoscope system 10 according to the present eleventh modification example, the image recognition unit 82B specifies the relationship between the traveling direction of the treatment tool and the papilla orientation ND. The derivation unit 82C generates the notification information 118 based on the positional relationship information 116A indicating the relationship between the traveling direction of the treatment tool and the papilla orientation ND. Accordingly, the user who observes the intestinal wall image 41 can be made to perceive the relationship between the traveling direction of the treatment tool and the papilla orientation ND.

In addition, in the above eleventh modification example, the form example in which the relationship between the traveling direction of the treatment tool and the papilla orientation ND is specified in the image recognition unit 82B has been described, but the technology of the present disclosure is not limited to this. For example, in the image recognition unit 82B, the relationship between the position of the papilla N and the position of the treatment tool may be specified together with the relationship between the traveling direction of the treatment tool and the papilla orientation ND. In this case, the positional relationship information 116A is information indicating the relationship between the traveling direction of the treatment tool and the papilla orientation ND and the relationship between the position of the papilla N and the position of the treatment tool, and the derivation unit 82C performs the determination related to the relationship between the traveling direction of the treatment tool and the papilla orientation ND and the determination related to the relationship between the position of the papilla N and the position of the treatment tool, based on the positional relationship information 116A. Moreover, the derivation unit 82C generates the notification information 118 according to these determination results.

### Twelfth Modification Example

In the above fourth embodiment, the form example in which the relationship between the position of the papilla N and the position of the treatment tool is specified as the positional relationship between the treatment tool and the papilla N has been described, but the technology of the present disclosure is not limited to this. In the present eleventh modification example, the relationship between the traveling direction of the treatment tool and the running direction TD of the bile duct is specified.

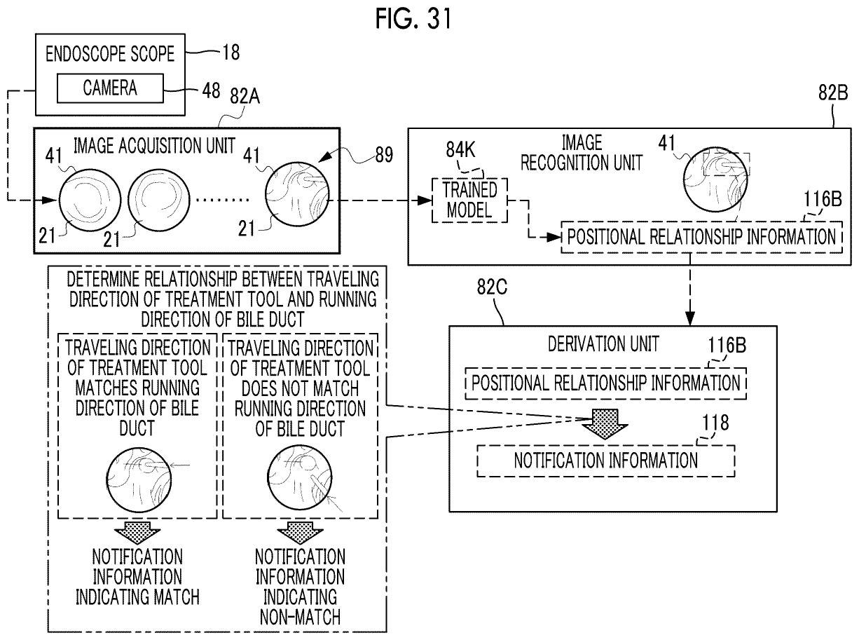

As shown in FIG. 31 as an example, the image recognition unit 82B acquires the time-series image group 89 from the image acquisition unit 82A and inputs the acquired time-series image group 89 to a trained model 84K. Accordingly, the trained model 84K outputs positional relationship information 116B corresponding to the input time-series image group 89. Here, the positional relationship information 116B is information (for example, an angle formed by the direction (hereinafter, simply referred to as a “bile duct tangential direction”) of a tangent line of an opening end part in the running direction TD of the bile duct and the traveling direction of the treatment tool) capable of specifying the relationship between the running direction TD of the bile duct and the traveling direction of the treatment tool.

The trained model 84K is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the relationship between the running direction TD of the bile duct and the traveling direction of the treatment tool.

The derivation unit 82C acquires the positional relationship information 116B from the image recognition unit 82B. The derivation unit 82C generates the notification information 118 that is information for notifying the user of the relationship between the running direction TD of the bile duct and the traveling direction of the treatment tool, based on the positional relationship information 116B. In a case where the angle between the bile duct tangential direction and the traveling direction of the treatment tool is within a predetermined range, the derivation unit 82C generates the notification information 118 indicating that the bile duct tangential direction matches the traveling direction of the treatment tool. In addition, in a case where the angle between the bile duct tangential direction and the traveling direction of the treatment tool exceeds a predetermined range, the derivation unit 82C generates the notification information 118 indicating that the bile duct tangential direction does not match the traveling direction of the treatment tool.

As described above, in the duodenoscope system 10 according to the present twelfth modification example, the image recognition unit 82B specifies the relationship between the traveling direction of the treatment tool and the running direction TD of the bile duct. The derivation unit 82C generates the notification information 118 based on the positional relationship information 116B indicating the relationship between the traveling direction of the treatment tool and the running direction TD of the bile duct. Accordingly, the user who observes the intestinal wall image 41 can be made to perceive the relationship between the traveling direction of the treatment tool and the running direction TD of the bile duct.

### Thirteenth Modification Example

In the above fourth embodiment, the form example in which the relationship between the position of the papilla N and the position of the treatment tool is specified as the positional relationship between the treatment tool and the papilla N has been described, but the technology of the present disclosure is not limited to this. In the present thirteenth modification example, a relationship between the traveling direction of the treatment tool and the orientation of a plane vertical to the rising direction RD of the papillary protuberance NA (hereinafter, also simply referred to as a “vertical plane orientation”) is specified.

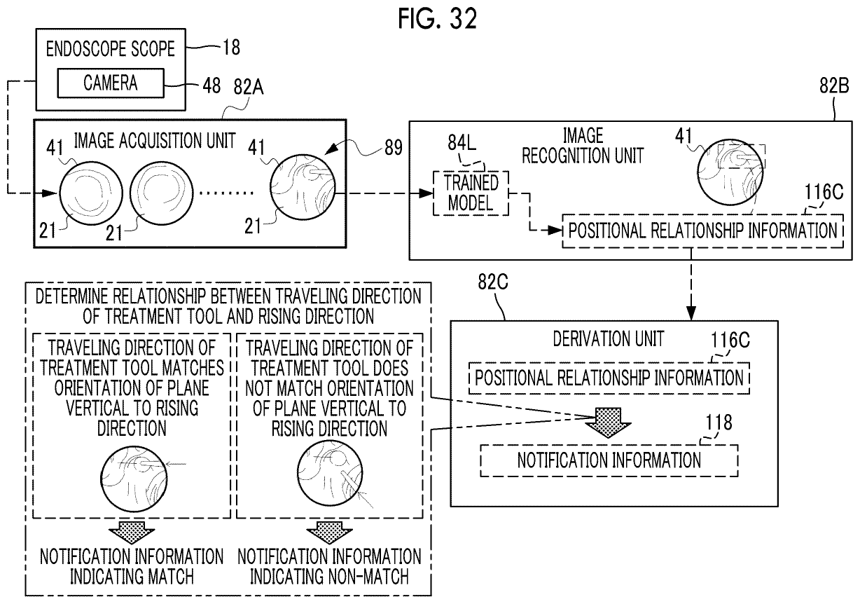

As shown in FIG. 32 as an example, the image recognition unit 82B acquires the time-series image group 89 from the image acquisition unit 82A and inputs the acquired time-series image group 89 to a trained model 84L. Accordingly, the trained model 84L outputs positional relationship information 116C corresponding to the input time-series image group 89. Here, the positional relationship information 116C is information (for example, an angle formed by the vertical plane orientation and the traveling direction of the treatment tool) capable of specifying the relationship between the vertical plane orientation and the traveling direction of the treatment tool.

The trained model 84L is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the relationship between the vertical plane orientation and the traveling direction of the treatment tool.

The derivation unit 82C acquires the positional relationship information 116C from the image recognition unit 82B. The derivation unit 82C generates the notification information 118 that is information for notifying the user of the relationship between the vertical plane orientation and the traveling direction of the treatment tool, based on the positional relationship information 116C. In a case where the angle between the vertical plane orientation and the traveling direction of the treatment tool is within a predetermined range, the derivation unit 82C generates the notification information 118 indicating that the vertical plane orientation matches the traveling direction of the treatment tool. In addition, in a case where the angle between the vertical plane orientation and the traveling direction of the treatment tool exceeds a predetermined range, the derivation unit 82C generates the notification information 118 indicating that the vertical plane orientation does not match the traveling direction of the treatment tool.

As described above, in the duodenoscope system 10 according to the present thirteenth modification example, the image recognition unit 82B specifies the relationship between the vertical plane orientation and the traveling direction of the treatment tool. The derivation unit 82C generates the notification information 118 based on the positional relationship information 116C indicating the relationship between the vertical plane orientation and the traveling direction of the treatment tool. Accordingly, the user who observes the intestinal wall image 41 can be made to perceive the relationship between the vertical plane orientation and the traveling direction of the treatment tool.

### Fourteenth Modification Example

In the above fourth embodiment, the form example in which the positional relationship between the treatment tool and the papilla N is specified by performing the image recognition processing on the intestinal wall image 41 example has been described, but the technology of the present disclosure is not limited to this. In the present fourteenth modification example, an evaluation value related to the positional relationship between the treatment tool and the papilla N is acquired by performing the image recognition processing on the intestinal wall image 41.

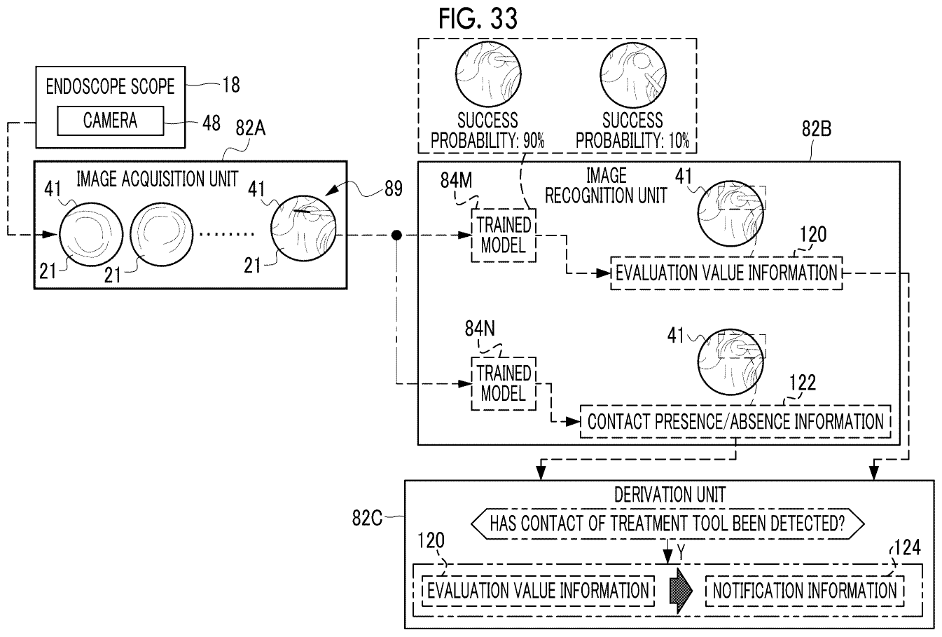

As shown in FIG. 33 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84M. Accordingly, the trained model 84M outputs evaluation value information 120 corresponding to the input time-series image group 89. The image recognition unit 82B acquires the evaluation value information 120 output from the trained model 84M. Here, the evaluation value information 120 is information (for example, the degree of success of a procedure determined according to the placement of the papilla N and the treatment tool) capable of specifying an evaluation value related to an appropriate placement of the papilla N and the treatment tool. The evaluation value information 120 is, for example, a plurality of scores (scores for each success or failure of the procedure) input to an activation function (for example, a softmax function or the like) of the output layer of the trained model 84M.

The trained model 84M is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data is an annotation (for example, an annotation indicating the success or failure of the procedure) capable of specifying an evaluation value related to an appropriate placement of the papilla N and the treatment tool.

In addition, the image recognition unit 82B inputs the time-series image group 89 to a trained model 84N. Accordingly, the trained model 84N outputs contact presence/absence information 122 corresponding to the input time-series image group 89. The image recognition unit 82B acquires the contact presence/absence information 122 output from the trained model 84N. Here, the contact presence/absence information 122 is information capable of specifying the presence or absence of contact between the papilla N and the treatment tool.

The trained model 84N is obtained by performing machine learning using training data on the neural network to optimize the neural network. The training data is a plurality of pieces of data (that is, a plurality of frames of data) in which example data and correct answer data are associated with each other. The example data is, for example, an image (for example, an image corresponding to the intestinal wall image 41) obtained by imaging a part (for example, an inner wall of the duodenum) that can be a target for the ERCP examination. The correct answer data is an annotation corresponding to the example data. An example of the correct answer data includes an annotation capable of specifying the presence or absence of contact between the papilla N and the treatment tool.

The derivation unit 82C acquires the contact presence/absence information 122 from the image recognition unit 82B. The derivation unit 82C determines whether or not the contact between the treatment tool and the papilla N is detected based on the contact presence/absence information 122. In a case where the contact between the treatment tool and the papilla N is detected, the derivation unit 82C generates notification information 124 based on the evaluation value information 120. The notification information 124 is information (for example, text indicating the success probability of the procedure) for notifying the user of the success probability of the procedure.

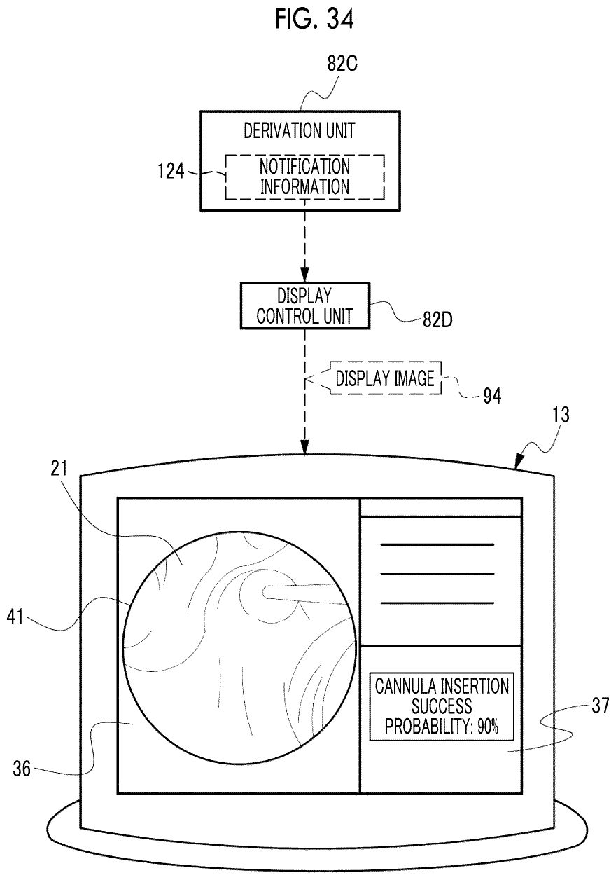

As shown in FIG. 34 as an example, the display control unit 82D acquires the notification information 124 from the derivation unit 82C. The derivation unit 82C outputs the notification information 124 to the display control unit 82D. In this case, the display control unit 82D generates the display image 94 including the content of notifying the user of the success probability of the procedure indicated by the notification information 124. In the example shown in FIG. 34, on the display device 13, a message “cannula insertion success probability: 90%” is displayed on the screen 37.

As described above, in the duodenoscope system 10 according to the present fourteenth modification example, the image recognition unit 82B of the processor 82 performs the image recognition processing on the intestinal wall image 41 and calculates the evaluation value related to the placement of the treatment tool and the papilla N. The derivation unit 82C generates the notification information 124 based on the evaluation value information 120 indicating the evaluation value. In the display control unit 82D, the display image 94 is generated based on the notification information 124 and is output to the display device 13. The display image 94 includes a display related to the success probability of the procedure indicated by the notification information 124. Accordingly, the user who observes the intestinal wall image 41 can be notified of the success probability of the procedure using the treatment tool. Since the user can consider the continuation or the change of the operation after grasping the success probability of the procedure, it is possible to support the success of the procedure using the treatment tool.

In addition, in the duodenoscope system 10 according to the present fourteenth modification example, the image recognition unit 82B performs the image recognition processing on the intestinal wall image 41 and specifies the presence or absence of contact between the treatment tool and the papilla N. Then, in a case where the treatment tool and the papilla N are in contact with each other based on the contact presence/absence information 122, the derivation unit 82C generates the notification information 124 based on the evaluation value information 120. Accordingly, the user who observes the intestinal wall image 41 can be notified of the success probability of the procedure using the treatment tool only in a necessary situation. In other words, it is possible to support the procedure for the papilla N using the treatment tool at an appropriate timing.

### Fifth Embodiment

In the above fourth embodiment, the form example in which the positional relationship between the treatment tool and the papilla N is specified by performing the image recognition processing on the intestinal wall image 41 has been described, but the technology of the present disclosure is not limited to this. In the present fifth embodiment, in a case where the treatment tool is an incision tool, an incision direction is obtained based on the result of the image recognition processing on the intestinal wall image 41.

For example, in the ERCP examination, an incision tool (for example, a papillotomy knife) as the treatment tool may be used. This is because the papilla N is incised using the incision tool, so that the insertion of the treatment tool into the papilla N is facilitated or foreign matter in the bile duct T or the pancreatic duct S is easily removed. In this case, in a case where the direction (that is, the incision direction) in which the papilla N is incised by the incision tool is erroneously selected, it may be difficult to perform the procedure successfully due to inadvertent bleeding or the like. Thus, in the present fifth embodiment, a direction (that is, an incision-recommended direction) recommended as the incision direction is specified by performing the image recognition processing on the intestinal wall image 41.

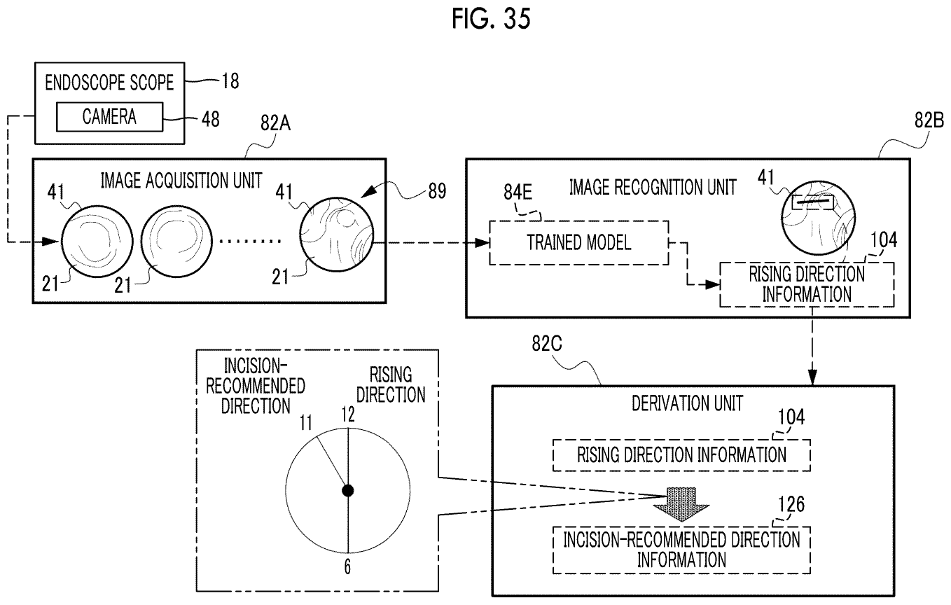

As shown in FIG. 35 as an example, the image acquisition unit 82A updates the time-series image group 89 using the FIFO method each time the intestinal wall image 41 is acquired from the camera 48.

The image recognition unit 82B acquires the time-series image group 89 from the image acquisition unit 82A and inputs the acquired time-series image group 89 to a trained model 84E. Accordingly, the trained model 84E outputs rising direction information 104 corresponding to the input time-series image group 89.

The derivation unit 82C acquires the rising direction information 104 from the image recognition unit 82B. Then, the derivation unit 82C derives incision-recommended direction information 126 based on the rising direction information 104. The incision-recommended direction information 126 is information (for example, a position coordinate group of a start point and an end point of the incision-recommended direction) capable of specifying the incision-recommended direction. The derivation unit 82C derives the incision-recommended direction from, for example, a predetermined orientation relationship between the rising direction RD and the incision-recommended direction. Specifically, the derivation unit 82C derives the incision-recommended direction as a direction of 11 o'clock in a case where the rising direction RD is a direction of 12 o'clock.

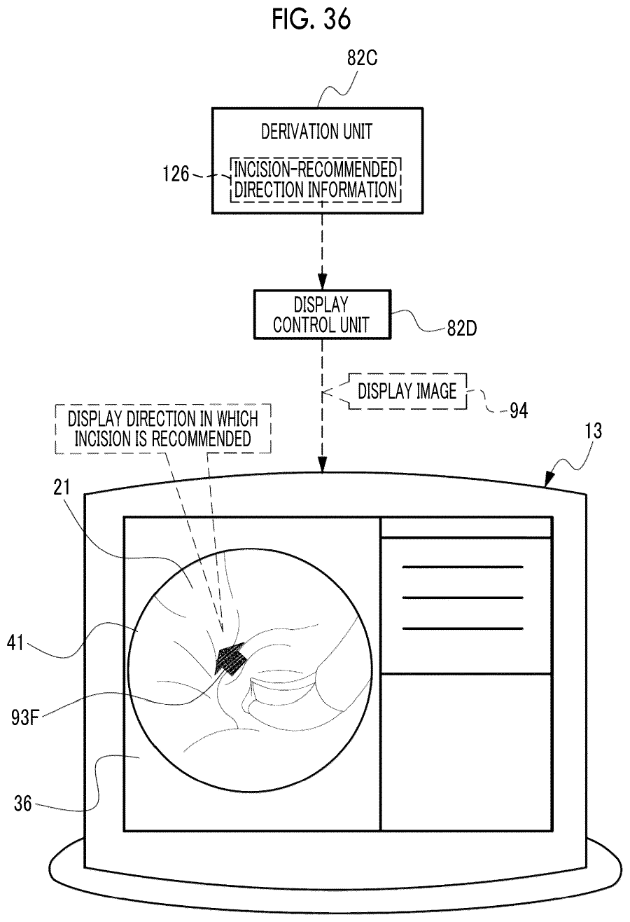

As shown in FIG. 36 as an example, the display control unit 82D acquires the incision-recommended direction information 126 from the derivation unit 82C. The display control unit 82D generates an incision direction image 93F, which is an image showing the incision direction, based on the incision-recommended direction indicated by the incision-recommended direction information 126. Then, the display control unit 82D generates the display image 94 including the incision direction image 93F and the intestinal wall image 41, and outputs the display image 94 to the display device 13. In the example shown in FIG. 36, the intestinal wall image 41 on which the incision direction image 93F is superimposed and displayed on the screen 36 is shown on the display device 13.

As described above, in the duodenoscope system 10 according to the present fifth embodiment, the incision-recommended direction information 126 is generated in the derivation unit 82C. The display control unit 82D generates the display image 94 based on the incision-recommended direction information 126 and outputs the display image 94 to the display device 13. The display image 94 includes the incision direction image 93F indicating the incision-recommended direction indicated by the incision-recommended direction information 126. Accordingly, the user who observes the intestinal wall image 41 can be made to grasp the incision-recommended direction. As a result, it is possible to support the success of the incision for the papilla N.

### Fifteenth Modification Example

In addition, in the above fifth embodiment, the form example in which the incision-recommended direction is specified has been described, but the technology of the present disclosure is not limited to this. In the present fifteenth modification example, a direction (that is, an incision non-recommended direction) that is not recommended as the incision direction may be specified.

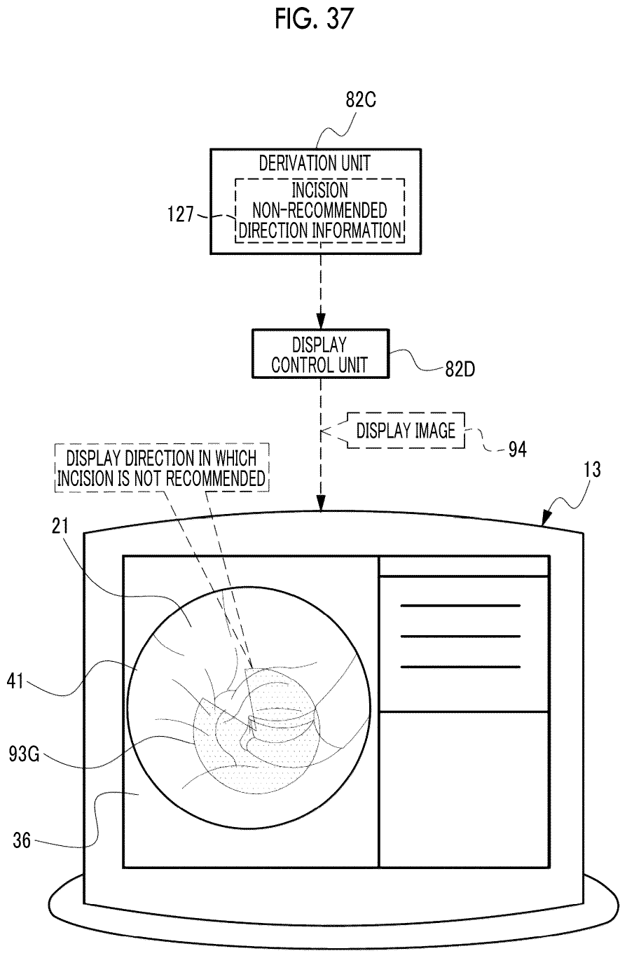

As shown in FIG. 37 as an example, the derivation unit 82C derives incision non-recommended direction information 127. The incision non-recommended direction information 127 is information (for example, an angle indicating a direction other than the incision-recommended direction) capable of specifying the incision non-recommended direction. The derivation unit 82C derives the incision-recommended direction from, for example, a predetermined orientation relationship between the rising direction RD and the incision-recommended direction. Specifically, the derivation unit 82C derives the incision-recommended direction as a direction of 11 o'clock in a case where the rising direction RD is a direction of 12 o'clock. Then, the derivation unit 82C specifies a range excluding a predetermined angle range (for example, a range of ±5 degrees centered on the incision-recommended direction) including the incision-recommended direction as the incision non-recommended direction.

The display control unit 82D acquires the incision non-recommended direction information 127 from the derivation unit 82C. The display control unit 82D generates an incision non-recommended direction image 93G, which is an image showing the incision non-recommended direction, based on the incision non-recommended direction indicated by the incision non-recommended direction information 127. Then, the display control unit 82D generates a display image 94 including the incision non-recommended direction image 93G and the intestinal wall image 41, and outputs the display image 94 to the display device 13. In the example shown in FIG. 37, the intestinal wall image 41 on which the incision non-recommended direction image 93G is superimposed and displayed on the screen 36 is shown on the display device 13.

As described above, in the duodenoscope system 10 according to the present fifteenth modification example, the derivation unit 82C generates the incision non-recommended direction information 127. The display control unit 82D generates the display image 94 based on the incision non-recommended direction information 127, and outputs the display image 94 to the display device 13. The display image 94 includes the incision non-recommended direction image 93G indicating the incision non-recommended direction indicated by the incision non-recommended direction information 127. Accordingly, the user who observes the intestinal wall image 41 can be made to grasp the incision non-recommended direction. As a result, it is possible to support the success of the incision for the papilla N.

In each of the above embodiments, as an aspect of indicating the operation direction to the user, the form example in which an image of an arrow indicating the operation direction is displayed on the screen 36 has been described, but the technology of the present disclosure is not limited to this. For example, the image indicating the operation direction may be an image of a triangle indicating the operation direction. In addition, an aspect in which a message indicating the operation direction is displayed instead of the image indicating the operation direction or together with the image may be adopted. Moreover, the image indicating the operation direction may be displayed on another window or another display device instead of being displayed on the screen 36.

In addition, in each of the above embodiments, the form example in which the bile duct direction TD is indicated has been described, but the technology of the present disclosure is not limited to this. An aspect in which the running direction of the pancreatic duct S is indicated instead of the bile duct direction TD or together with the bile duct direction TD may be adopted.

In addition, in each of the above embodiments, the form example in which the various types of information are output to the display device 13 has been described, but the technology of the present disclosure is not limited to this. For example, the various types of information may be output to a voice output device such as a speaker (not shown) instead of the display device 13 or together with the display device 13, or may be output to a printing device such as a printer (not shown).

In each of the above embodiments, the form example in which the various types of information are output to the display device 13 and the information is displayed on the screen 36 of the display device 13 has been described, but the technology of the present disclosure is not limited to this. The various types of information may be output to an electronic medical record server. The electronic medical record server is a server that stores electronic medical record information indicating a medical care result for a patient. The electronic medical record information includes the various types of information.

The electronic medical record server is connected to the duodenoscope system 10 through a network. The electronic medical record server acquires the intestinal wall image 41 and the various types of information from the duodenoscope system 10. The electronic medical record server stores the intestinal wall image 41 and the various types of information as a part of the medical care result indicated by the electronic medical record information.

The electronic medical record server is also connected to a terminal (for example, a personal computer installed in a medical care facility) other than the duodenoscope system 10 through a network. The user, such as the doctor 14, can obtain the intestinal wall image 41 and the various types of information stored in the electronic medical record server through the terminal. In this way, since the intestinal wall image 41 and the various types of information are stored in the electronic medical record server, the user can obtain the intestinal wall image 41 and the various types of information.

In addition, in each of the above embodiments, the form example in which the image recognition processing using the AI method is executed on the intestinal wall image 41 has been described, but the technology of the present disclosure is not limited to this. For example, the image recognition processing using the pattern matching method may be executed.

In the above embodiment, the form example in which the medical support processing is performed by the processor 82 of the computer 76 included in the image processing device 25 has been described, but the technology of the present disclosure is not limited to this. For example, the medical support processing may be performed by the processor 70 of the computer 64 included in the control device 22. In addition, a device that performs the medical support processing may be provided outside the duodenoscope 12. An example of the device provided outside the duodenoscope 12 is at least one server and/or at least one personal computer that is communicably connected to the duodenoscope 12. In addition, the medical support processing may be performed in a distributed manner by a plurality of devices.

In the above embodiment, the form example in which the medical support processing program 84A is stored in the NVM 84 has been described, but the technology of the present disclosure is not limited to this. For example, the medical support processing program 84A may be stored in a portable non-transitory storage medium such as an SSD or a USB memory. The medical support processing program 84A stored in the non-transitory storage medium is installed in the computer 76 of the duodenoscope 12. The processor 82 performs the medical support processing according to the medical support processing program 84A.

In addition, the medical support processing program 84A may be stored in a storage device of, for example, another computer or a server that is connected to the duodenoscope 12 through a network. Then, the medical support processing program 84A may be downloaded and installed in the computer 76 in response to a request from the duodenoscope 12.

It is not necessary to store all of the medical support processing program 84A in the NVM 84 or the storage device of another computer or server device connected to the duodenoscope 12, and a part of the medical support processing program 84A may be stored.

Various processors described below can be used as the hardware resource for executing the medical support processing. An example of the processor is a CPU which is a general-purpose processor that executes software, that is, a program, to function as the hardware resource performing the medical support processing. In addition, an example of the processor is a dedicated electronic circuit which is a processor having a dedicated circuit configuration designed to perform a specific process, such as an FPGA, a PLD, or an ASIC. Any processor has a memory built in or connected to it, and any processor executes the medical support processing by using the memory.

The hardware resource for performing the medical support processing may be configured by one of the various processors or by a combination of two or more processors of the same type or different types (for example, a combination of a plurality of FPGAs or a combination of a CPU and an FPGA). The hardware resource for executing the medical support processing may also be one processor.

A first example of the configuration using one processor is a form in which one processor is configured by combining one or more CPUs and software, and the processor functions as the hardware resource for executing the medical support processing. A second example of the configuration is an aspect in which a processor that implements the functions of the entire system including a plurality of hardware resources for performing the medical support processing using one IC chip is used. A representative example of this aspect is an SoC. In this way, the medical support processing is implemented by using one or more of the various processors as the hardware resource.

More specifically, an electric circuit in which circuit elements such as semiconductor elements are combined can be used as a hardware structure of the various processors. In addition, the above medical support processing is merely an example. Therefore, it goes without saying that unnecessary steps may be deleted, new steps may be added, and the processing order may be changed within a range that does not deviate from the scope.

The above-described contents and the above-shown contents are detailed descriptions of portions relating to the technology of the present disclosure and are merely examples of the technology of the present disclosure. For example, the description of the configuration, the function, the operation, and the effect above are the description of examples of the configuration, the function, the operation, and the effect of the parts according to the technology of the present disclosure. As a result, it goes without saying that unnecessary parts may be deleted, new elements may be added, or replacements may be made with respect to the above-described contents and the above-shown contents within a range that does not deviate from the gist of the technology of the present disclosure. In addition, the description of, for example, common technical knowledge that does not need to be particularly described to enable the implementation of the technology of the present disclosure is omitted in the above-described contents and the above-shown contents in order to avoid confusion and to facilitate understanding of the portions relating to the technology of the present disclosure.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All documents, patent applications, and technical standards described in the present specification are incorporated in the present specification by reference in their entireties to the same extent as in a case where the individual documents, patent applications, and technical standards are specifically and individually written to be incorporated by reference.

The disclosure of JP2022-177613 filed on Nov. 4, 2022 is incorporated in the present specification by reference.

What is claimed is:

1. A medical support device comprising:
a processor,
wherein the processor is configured to:
acquire intestinal tract direction-related information related to an intestinal tract direction of a duodenum into which an endoscope scope is inserted, based on geometrical characteristic information capable of specifying a geometrical characteristic of the duodenum; and
output the intestinal tract direction-related information.

2. The medical support device according to claim 1,
wherein the intestinal tract direction-related information includes deviation amount information indicating a deviation amount between a posture of the endoscope scope and the intestinal tract direction.

3. The medical support device according to claim 1,
wherein the geometrical characteristic information includes an intestinal wall image obtained by imaging an intestinal wall of the duodenum with a camera provided in the endoscope scope, and
the processor is configured to acquire the intestinal tract direction-related information by executing first image recognition processing on the intestinal wall image.

4. The medical support device according to claim 1,
wherein the outputting of the intestinal tract direction-related information includes displaying the intestinal tract direction-related information on a first screen.

5. The medical support device according to claim 1,
wherein the intestinal tract direction-related information includes first direction information capable of specifying a first direction intersecting the intestinal tract direction at a predetermined angle.

6. The medical support device according to claim 5,
wherein the first direction information is obtained by executing second image recognition processing on an intestinal wall image obtained by imaging an intestinal wall of the duodenum with a camera provided in the endoscope scope.

7. The medical support device according to claim 6,
wherein the first direction information is information obtained with a degree of certainty equal to or greater than a threshold value by performing image recognition processing using an AI method as the second image recognition processing.

8. The medical support device according to claim 1,
wherein the processor is configured to acquire posture information capable of specifying a posture of the endoscope scope in a state in which the endoscope scope is inserted into the duodenum,
the intestinal tract direction-related information includes posture adjustment support information for supporting adjustment of the posture, and
the posture adjustment support information is information determined based on a deviation amount between the intestinal tract direction and the posture specified from the posture information.

9. The medical support device according to claim 1,
wherein the intestinal tract direction-related information includes condition information indicating a condition for changing a posture of the endoscope scope to match an optical axis direction of a camera provided in the endoscope scope with a second direction intersecting the intestinal tract direction at a predetermined angle.

10. The medical support device according to claim 9,
wherein the condition includes an operation condition related to an operation performed on the endoscope scope to match the optical axis direction with the second direction.

11. The medical support device according to claim 1,
wherein, in a case where an optical axis direction of a camera provided in the endoscope scope matches a third direction intersecting the intestinal tract direction at a predetermined angle, the intestinal tract direction-related information includes notification information for notifying that the optical axis direction matches the third direction.

12. The medical support device according to claim 1,
wherein the processor is configured to:
detect a duodenal papilla region by executing third image recognition processing on an intestinal wall image obtained by imaging an intestinal wall of the duodenum with a camera provided in the endoscope scope;
display the duodenal papilla region on a second screen; and
display, on the second screen, papilla orientation information indicating an orientation of the duodenal papilla region, the papilla orientation information being obtained based on the intestinal tract direction-related information.

13. The medical support device according to claim 1,
wherein the processor is configured to:
detect a duodenal papilla region by executing fourth image recognition processing on an intestinal wall image obtained by imaging an intestinal wall of the duodenum with a camera provided in the endoscope scope;
display the duodenal papilla region on a third screen; and
display, on the third screen, running direction information indicating a running direction of a duct leading to an opening of the duodenal papilla region, the running direction information being obtained based on the intestinal tract direction-related information.

14. The medical support device according to claim 13,
wherein the duct is a bile duct or a pancreatic duct.

15. The medical support device according to claim 1,
wherein the geometrical characteristic information includes depth information indicating a depth of the duodenum, and
the intestinal tract direction-related information is acquired based on the depth information.

16. An endoscope comprising:
the medical support device according to claim 1; and
the endoscope scope.

17. A medical support method comprising:
acquiring intestinal tract direction-related information related to an intestinal tract direction of a duodenum into which an endoscope scope is inserted, based on geometrical characteristic information capable of specifying a geometrical characteristic of the duodenum; and
outputting the intestinal tract direction-related information.

18. A non-transitory computer-readable storage medium storing a program causing a computer to execute processing comprising:
acquiring intestinal tract direction-related information related to an intestinal tract direction of a duodenum into which an endoscope scope is inserted, based on geometrical characteristic information capable of specifying a geometrical characteristic of the duodenum; and
outputting the intestinal tract direction-related information.
